(19) United States
(12) Patent Application Publication
(10) Pub. No.: US 2024/0363144 A1
(43) Pub. Date: Oct. 31, 2024

(54) COMPOUND, OPTICAL ABSORBING MATERIAL, NON-LINEAR OPTICAL ABSORBING MATERIAL, RECORDING MEDIUM, INFORMATION RECORDING METHOD, AND INFORMATION READING METHOD

(71) Applicant: Panasonic Intellectual Property Management Co., Ltd., Osaka (JP)

(72) Inventors: MASAKO YOKOYAMA, Osaka (JP); KOTA ANDO, Osaka (JP); HIDEKAZU ARASE, Hyogo (JP)

(21) Appl. No.: 18/766,750

(22) Filed: Jul. 9, 2024

Related U.S. Application Data
(63) Continuation of application No. PCT/JP2022/046921, filed on Dec. 20, 2022.

(30) Foreign Application Priority Data
Jan. 24, 2022 (JP) 2022-008998

(51) Int. Cl. G11B 7/0045; C07C 15/54; C07C 25/24; C07F 5/00; C07F 7/08; C09K 11/06; G02F 1/361; G11B 7/005; G11B 7/246; G11B 7/249

(52) U.S. Cl. CPC G11B 7/00455; C07C 15/54; C07C 25/24; C07F 5/00; C07F 7/0805; C09K 11/06; G02F 1/3619; G11B 7/0052; G11B 7/246; G11B 7/249; G11B 2007/24624

(57) ABSTRACT

A compound is represented by Formula (1) below,

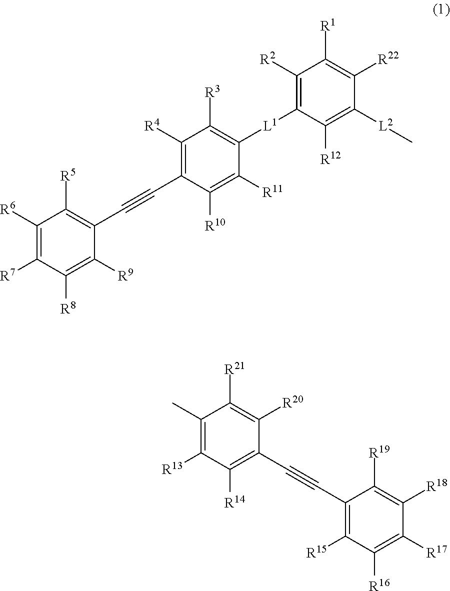

in Formula (1) above, R¹ to R²² each independently contain at least one atom selected from the group consisting of H, B, C, N, O, F, Si, P, S, Cl, I, and Br, and L¹ and L² each independently represent a single bond or —C≡C—.

### BACKGROUND

### 1. Technical Field

The present disclosure relates to a compound, an optical absorbing material, a non-linear optical absorbing material, a recording medium, an information recording method, and an information reading method.

### 2. Description of the Related Art

Of optical materials such as optical absorbing materials, a material having a non-linear optical effect is referred to as a non-linear optical material. The non-linear optical effect means that when a substance is irradiated with intense light such as laser light, an optical phenomenon proportionate to the electric field of the applied light to the power of 2 or higher occurs in the substance. Examples of the phenomenon include absorption, reflection, scattering, and light emission. Examples of the second order non-linear optical effect proportionate to the electric field of the applied light to the power of 2 include second harmonic generation (SHG), Pockels effect, and a parametric effect. Examples of the third order non-linear optical effect proportionate to the electric field of the applied light to the power of 3 include two-photon absorption, multiphoton absorption, third-harmonic generation (THG), and Kerr effect. In the present specification, multiphoton absorption such as two-photon absorption is also referred to as non-linear optical absorption. A material capable of performing non-linear optical absorption is also referred to as a non-linear optical absorbing material. In particular, a material capable of performing two-photon absorption is also referred to as a two-photon-absorbing material. In this regard, non-linear optical absorption is also referred to as non-linear absorption.

To date, considerable research has been intensively performed on non-linear optical materials. In particular, inorganic materials capable of readily preparing a single crystal have been developed as non-linear optical materials. In recent years, development of a non-linear optical material composed of an organic material has been desired. An organic material has not only a high degree of design flexibility but also a large non-linear optical constant compared with an inorganic material. Further, regarding an organic material, non-linear response is performed at a high speed. In the present specification, a non-linear optical material containing an organic material is also referred to as an organic non-linear optical material.

### SUMMARY

In one general aspect, the techniques disclosed here feature a compound represented by Formula (1) below,

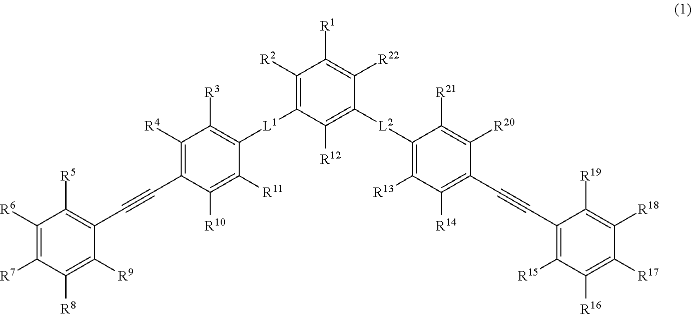

in Formula (1) above, R¹ to R²² each independently contain at least one atom selected from the group consisting of H, B, C, N, O, F, Si, P, 5, Cl, I, and Br, L¹ and L² each independently represent a single bond or —C≡C—, R¹, R², R⁶, R⁷, R¹², R¹⁷, and R²² are each independently a substituent other than a substituent having an aromatic ring, and R¹ to R²² are each independently a hydrogen atom or a substituent having a Hammett substituent constant σₚ within a range of greater than or equal to −0.2 and less than or equal to 0.2.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a flow chart illustrating an information recording method by using a recording medium containing a compound according to an embodiment of the present disclosure;

FIG. 1B is a flow chart illustrating an information reading method by using a recording medium containing a compound according to an embodiment of the present disclosure;

FIG. 2 is a graph illustrating a ¹H-NMR spectrum of Compound (2)-1; and

FIG. 3 is a graph illustrating a ¹H-NMR spectrum of Compound (3)-1.

### DETAILED DESCRIPTIONS

Underlying knowledge forming basis of the present disclosure

Regarding organic non-linear optical materials, two-photon-absorbing materials have particularly attracted attention. Two-photon absorption means a phenomenon in which a compound almost simultaneously absorbs two photons and transits to an excited state. Known examples of the two-photon absorption include simultaneous two-photon absorption and stepwise two-photon absorption. The simultaneous two-photon absorption is also referred to as nonresonant two-photon absorption. The simultaneous two-photon absorption means two-photon absorption in a wavelength range in which an absorption band of a single photon is not present. The stepwise two-photon absorption is also referred to as resonant two-photon absorption. In the stepwise two-photon absorption, a compound transits to a higher-order excited state by further absorbing a second photon after absorbing a first photon. Regarding the stepwise two-photon absorption, a compound successively absorbs two photons.

In the simultaneous two-photon absorption, usually, the amount of the light absorbed by a compound is proportionate to the irradiation light intensity to the power of 2 and exhibits nonlinearity. The amount of the light absorbed by a compound can be utilized as an indicator of the efficiency of the two-photon absorption. When the amount of the light absorbed by a compound exhibits nonlinearity, for example, the absorption of the light by the compound can be caused only around the focal point of the laser light having high electric field intensity. That is, in a sample containing a two-photon-absorbing material, the compound can be excited at only a predetermined position. As described above, since the compound in which two-photon absorption occurs provides very high spatial resolution, application of the compound to use such as a recording layer of a three-dimensional optical memory, a photo-curable resin composition for three-dimensional (3D) laser microfabrication, and the like has been researched. When the two-photon-absorbing material further has fluorescent characteristics, the two-photon-absorbing material can also be applied to a fluorescent coloring material used for two-photon fluorescent microscope and the like. When the two-photon-absorbing material is used for the three-dimensional optical memory, a system in which an ON/OFF state of the recording layer is read in accordance with a change in the fluorescence from the two-photon-absorbing material may be adopted. Regarding the current optical memory, a system in which an ON/OFF state of the recording layer is read in accordance with a change in the light reflectance and a change in the light absorptance in the two-photon-absorbing material is adopted. However, when this system is applied to the three-dimensional optical memory, since a two-photon-absorbing material in the related art has a low two-photon absorptance compared with a single-photon absorptance, crosstalk may occur due to another recording layer different from the recording layer, the ON/OFF state of which should be read.

Regarding the two-photon-absorbing material, a two-photon absorption cross-sectional area (GM value) is used as an indicator for indicating the efficiency of the two-photon absorption. The unit of the two-photon absorption cross-sectional area is GM (10⁻⁵⁰ cm⁴·s·molecule·¹ photon·¹). To date, many organic two-photon-absorbing materials having a large two-photon absorption cross-sectional area have been proposed. For example, many compounds having such a large two-photon absorption cross-sectional area that is greater than 500 GM have been reported (for example, Harry L. Anderson et al, “Two-Photon Absorption and the Design of Two-Photon Dyes”, Angew. Chem. Int. Ed. 2009, Vol. 48, p. 3244-3266). In this regard, in most reports, the two-photon absorption cross-sectional area is measured using laser light having a wavelength of greater than 600 nm. In particular, near infrared rays having a wavelength greater than 750 nm may be used.

However, to apply the two-photon-absorbing material to industrial use, a material that realizes two-photon absorption characteristics when irradiated with laser light having a shorter wavelength is required. For example, in the field of the three-dimensional optical memory, the laser light having a short wavelength can improve the recording density of the three-dimensional optical memory since a finer condensed light spot can be realized. In the field of the three-dimensional (3D) laser microfabrication, the laser light having a short wavelength can realize shaping at higher resolution. Further, according to the Blu-ray® disc standard, laser light having a center wavelength of 405 nm is used. Consequently, development of a compound having excellent two-photon absorption characteristics with respect to light in the same wavelength range as the laser light having a short wavelength can significantly contribute to industrial development.

Further, light-emitting apparatuses which emit ultrashort pulse laser having high light intensity are large, and actions tend to be unstable. Therefore, it is difficult to adopt such light-emitting apparatuses for industrial use from the viewpoint of universality and reliability. In consideration of this, to apply the two-photon-absorbing material to industrial use, a material that realizes two-photon absorption characteristics even when laser light having low light intensity is applied is required.

Regarding the compound having two-photon absorption characteristics, the relationship between the light intensity and the two-photon absorption characteristics is denoted by Formula (i) below. In the present specification, a compound having two-photon absorption characteristics is also referred to as a two-photon-absorbing compound. Formula (i) is a calculation formula for calculating a decrease in light intensity −dI when light having intensity I is applied to a sample containing a two-photon-absorbing compound and having an infinitesimal thickness dz. As is clear from Formula (i), the decrease in light intensity −dI is expressed by the sum of a term proportionate to the incident light intensity I to the power of 1 and a term proportionate to the intensity I to the power of 2.

$$- \frac{dI}{dz} = α I + {α}^{( 2 )} {I}^{2} \qquad ( i )$$

In Formula (i), α represents a single-photon absorption coefficient (cm⁻¹), and α⁽²⁾ represents a two-photon absorption coefficient (cm/W). It is clear from Formula (i) that the incident light intensity I is denoted by α/α⁽²⁾ when the amount of single-photon absorption is equal to the amount of two-photon absorption in a sample. That is, when the incident light intensity I is less than α/α⁽²⁾, single-photon absorption preferentially occurs in the sample. When the incident light intensity I is greater than α/α⁽²⁾, two-photon absorption preferentially occurs in the sample. Consequently, there is a tendency that two-photon absorption can be preferentially realized due to laser light having low light intensity with the value of α/α⁽²⁾ decreasing in the sample.

Further, α and α⁽²⁾ can be denoted by Formula (ii) and Formula (iii) below, respectively. In Formula (ii) and Formula (iii), ε represents a molar extinction coefficient (mol⁻¹·L·cm⁻¹). N represents the number of compound molecules per unit volume of a sample (mol·cm³). N_{A} represents Avogadro constant. σ represents a two-photon absorption cross-sectional area (GM). h− (h bar) represents Dirac constant (J·s). ω represents an angular frequency (rad/sec) of incident light.

$$α = 1000 \cdot ln 10 \cdot ε \cdot \frac{N}{{N}_{A}} \qquad ( ii )$$

$${α}^{( 2 )} = \frac{σ N}{ℏ ω} \qquad ( iii )$$

It is clear from Formula (ii) and Formula (iii) that α/α⁽²⁾ is determined by ε/σ. That is, to preferentially realize two-photon absorption due to laser light having low light intensity, it is desirable that the ratio σ/ε of the two-photon absorption cross-sectional area σ to the molar extinction coefficient F be large with respect to the wavelength of the applied laser light. Regarding the compound, when the value of the ratio σ/ε is large at a specific wavelength, it can be said that the non-linearity of light absorption is high at the wavelength.

Japanese Patent No. 5769151 and Japanese Patent No. 5659189 disclose compounds having a large two-photon absorption cross-sectional area with respect to the light having a wavelength around 405 nm. Japanese Patent No. 5821661 discloses an optical information recording medium capable of decreasing a writing time when the laser light having a wavelength around 405 nm is used and a compound contained in the optical information recording medium.

Japanese Patent No. 5769151 and Japanese Patent No. 5821661 describe compounds having a large π-electron conjugated system. Further, Japanese Patent No. 5659189 describes a benzophenone derivative having a large π-electron conjugated system. However, in the compound, when the π-electron conjugated system extends, a two-photon absorption cross-sectional area increases, whereas a peak due to single-photon absorption tends to shift to a long wavelength range. In the present specification, shift of a peak due to single-photon absorption to a long wavelength range is also referred to as a long wavelength shift or a red shift. As a result of the shift of a peak due to single-photon absorption to a long wavelength, a portion of a wavelength range in which single-photon absorption occurs may overlap the wavelength of the excitation light. In this regard, specific examples of the wavelength of an excitation light include 405 nm specified in the Blu-ray (registered trademark) standard. In the compound, when single-photon absorption due to the excitation light is large, the non-linearity of light absorption tends to be lowered. The compound having low non-linearity of light absorption is unsuitable for the recording layer of a multilayered three-dimensional optical memory.

Further, regarding the benzophenone derivative disclosed in Japanese Patent No. 5659189, the quantum yield of the intersystem crossing is almost 100%. Since this benzophenone derivative promptly transits from a singlet excited state to a triplet excited state, almost no fluorescence is emitted.

The present inventors performed intensive research and, as a result, newly found that the compound denoted by Formula (1) later has high non-linear optical absorption characteristics with respect to the light having a wavelength in a short wavelength range. In particular, the present inventors found that the compound denoted by Formula (1) tends to have a large value of ratio σ/ε of the two-photon absorption cross-sectional area a to the molar extinction coefficient F with respect to the light having a wavelength in a short wavelength range and tends to have high non-linearity of light absorption. Further, this compound tends to have also fluorescent characteristics. In the present specification, the short wavelength range means a wavelength range including 405 nm and means, for example, a wavelength range of greater than or equal to 390 nm and less than or equal to 420 nm.

Outline of aspect according to the present disclosure

A compound according to a first aspect of the present disclosure is represented by Formula (1) below,

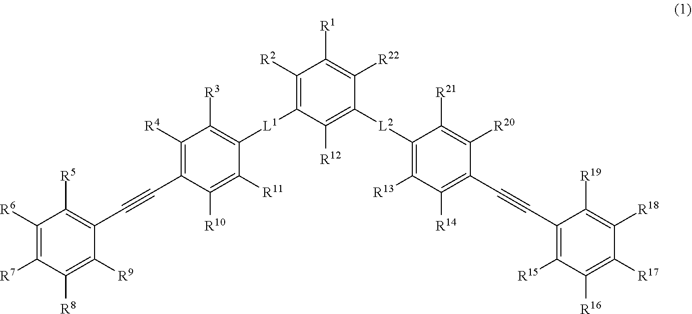

in Formula (1) above, R¹ to R²² each independently contain at least one atom selected from the group consisting of H, B, C, N, O, F, Si, P, S, Cl, I, and Br and L¹ and L² each independently represent a single bond or —C≡C—.

In this regard, the compound satisfies the following requirements (a) and (b).
(a) R¹, R², R⁶, R⁷, R¹², R¹⁷, and R²² are each independently a substituent other than a substituent having an aromatic ring.
(b) R¹ to R²² are each independently a hydrogen atom or a substituent having Hammett substituent constant 6p within the range of greater than or equal to −0.2 and less than or equal to 0.2.

The compound according to the first aspect tends to have a large ratio σ/ε of the two-photon absorption cross-sectional area a to the molar extinction coefficient F with respect to the light having a wavelength in a short wavelength range and have high non-linearity of light absorption. Consequently, regarding the composition, the non-linear optical absorption characteristics with respect to the light having a wavelength in a short wavelength range are improved. The compound according to the first aspect also tends to have fluorescent characteristics.

According to the requirement (b), even when each of R¹ to R²² has a substituent, since the substituent constant σₚ of the substituent is a value close to 0, the electron-withdrawing property or the electron-donating property of the substituent is low.

Consequently, an increase in the energy of the highest occupied molecular orbital (HOMO) and a decrease in the energy of the lowest unoccupied molecular orbital (LUMO) of the compound due to the electron-withdrawing property or the electron-donating property of the substituent can be suppressed from occurring. That is, the energy gap between HOMO and LUMO can be suppressed from decreasing. Consequently, the peak due to single-photon absorption can be suppressed from shifting to a long wavelength, and the ratio σ/ε with respect to the light having a wavelength in a short wavelength range can be suppressed from decreasing. The compound satisfying the requirement (b) tends to have high non-linearity of light absorption with respect to the light having a wavelength in a short wavelength range.

In a second aspect of the present disclosure, for example, the compound according to the first aspect may be represented by Formula (2) below.

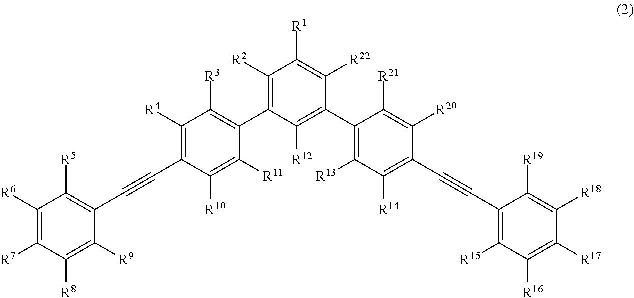

In a third aspect of the present disclosure, for example, the compound according to the first aspect may be represented by Formula (3) below.

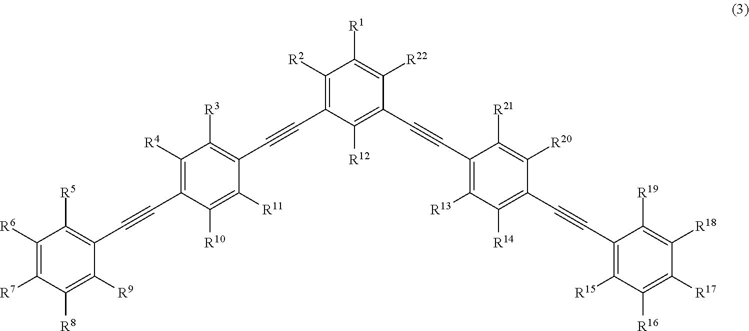

In a fourth aspect of the present disclosure, for example, in the compound according to any one of the first aspect to the third aspect, R¹ to R²² above may be each independently a hydrogen atom, a halogen atom, a hydrocarbon group, a halogenated hydrocarbon group, a substituent containing an oxygen atom, a substituent containing a nitrogen atom, a substituent containing a sulfur atom, a substituent containing a silicon atom, a substituent containing a phosphorus atom, or a substituent containing a boron atom.

According to the second aspect to the fourth aspect, regarding the compound, the non-linear optical absorption characteristics with respect to the light having a wavelength in a short wavelength range are improved. The compounds according to the second aspect to the fourth aspect also tend to have fluorescent characteristics. These compounds are suitable for a device use in which the light having a wavelength of greater than or equal to 390 nm and less than or equal to 420 nm is utilized.

In a fifth aspect of the present disclosure, for example, in the compound according to any one of the first aspect to the fourth aspect, R¹ to R²² may be each independently a hydrogen atom.

The compound according to the fifth aspect tends to have higher non-linearity of light absorption with respect to the light having a wavelength in a short wavelength range.

In a sixth aspect of the present disclosure, for example, the compound according to any one of the first aspect to the fifth aspect may be used for a device that utilizes light having a wavelength of greater than or equal to 390 nm and less than or equal to 420 nm.

According to the sixth aspect, the compound is suitable for a device use in which the light having a wavelength of greater than or equal to 390 nm and less than or equal to 420 nm is utilized.

An optical absorbing material according to a seventh aspect of the present disclosure contains the compound according to any one of the first aspect to the sixth aspect.

According to the seventh aspect, regarding the optical absorbing material, the non-linear optical absorption characteristics with respect to the light having a wavelength in a short wavelength range are improved.

A non-linear optical absorbing material according to an eighth aspect of the present disclosure contains the compound according to any one of the first aspect to the sixth aspect.

According to the eighth aspect, regarding the non-linear optical absorbing material, the non-linear optical absorption characteristics with respect to the light having a wavelength in a short wavelength range are improved.

A recording medium according to a ninth aspect of the present disclosure includes a recording layer containing the compound according to any one of the first aspect to the sixth aspect.

According to the ninth aspect of the present disclosure, regarding the compound, the non-linear optical absorption characteristics with respect to the light having a wavelength in a short wavelength range are improved. The compound used in the ninth aspect also tends to have fluorescent characteristics. The recording medium including the recording layer containing such a compound can record information at a high recording density.

An information recording method according to a tenth aspect of the present disclosure includes
preparing a light source that emits light having a wavelength of greater than or equal to 390 nm and less than or equal to 420 nm, and
condensing the light from the light source and applying the light to the recording layer of the recording medium according to the ninth aspect.

According the tenth aspect, regarding the compound, the non-linear optical absorption characteristics with respect to the light having a wavelength in a short wavelength range are improved. The compound used in the tenth aspect also tends to have fluorescent characteristics. According to the information recording method by using the recording medium containing such a compound, the information can be recorded at a high recording density.

An information reading method according to an eleventh aspect is, for example, a method for reading the information recorded by the recording method according to the tenth aspect, and includes
measuring an optical characteristic of the recording layer by applying light to the recording layer of the recording medium, and
reading the information from the recording layer.

In a twelfth aspect according to the present disclosure, for example, regarding the information reading method according to the eleventh aspect,
the optical characteristic may be intensity of fluorescence emitted from the recording layer.

According to the eleventh aspect and the twelfth aspect, when the information is read, crosstalk due to another recording layer can be suppressed from occurring.

The embodiment according to the present disclosure will be described below with reference to the drawings. The present disclosure is not limited to the following embodiment.

### Embodiment

Compound A according to the present embodiment is denoted by Formula (1) below.

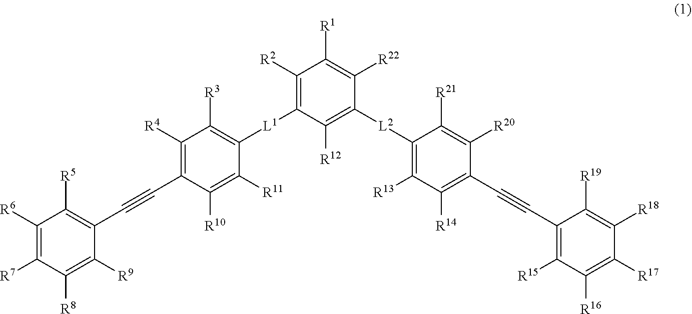

In Formula (1), R¹ to R²² each independently contain at least one atom selected from the group consisting of H, B, C, N, O, F, Si, P, S, Cl, I, and Br. R¹ to R²² may be each independently a hydrogen atom, a halogen atom, a hydrocarbon group, a halogenated hydrocarbon group, an oxygen-atom-containing substituent, a nitrogen-atom-containing substituent, a sulfur-atom-containing substituent, a silicon-atom-containing substituent, a phosphorus-atom-containing substituent, or a boron-atom-containing substituent.

Examples of the halogen atom include F, Cl, Br, and I. In the present specification, a halogen atom is also referred to as a halogen group.

There is no particular limitation regarding the carbon number of the hydrocarbon group, and the carbon number is, for example, greater than or equal to 1 and less than or equal to 20, may be greater than or equal to 1 and less than or equal to 10, or may be greater than or equal to 1 and less than or equal to 5. The solvent or resin composition solubility of Compound A can be adjusted by adjusting the carbon number of the hydrocarbon group. The hydrocarbon group may have the shape of a strait chain, a branched chain, or a ring.

Examples of the hydrocarbon group include aliphatic saturated hydrocarbon groups, alicyclic hydrocarbon groups, and aliphatic unsaturated hydrocarbon groups. The aliphatic saturated hydrocarbon group may be an alkyl group. Examples of the aliphatic saturated hydrocarbon group include —CH₃, —CH₂CH₃, —CH₂CH₂CH₃, —CH(CH₃)₂, —CH(CH₃)CH₂CH₃, —C(CH₃)₃, —CH₂CH(CH₃)₂, —(CH₂)₃CH₃, —(CH₂)₄CH₃, —C(CH₂CH₃)(CH₃)₂, —CH₂C(CH₃)₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, —(CH₂)₁₄CH₃, —(CH₂)₁₅CH₃, —(CH₂)₁₆CH₃, —(CH₂)₁₇CH₃, —(CH₂)₁₈CH₃, and —(CH₂)₁₉CH₃. Examples of the alicyclic hydrocarbon group include a cyclopropyl group, a cyclobutyl group, a cyclopentyl group, a cyclohexyl group, and an adamantyl group. Examples of the aliphatic unsaturated hydrocarbon group include —CH═CH₂, —C≡CH, —C≡CCH₃, —C(CH₃)═CH₂, —CH═CHCH₃, and —CH₂CH═CH₂.

The halogenated hydrocarbon group means a group in which at least one hydrogen atom included in a hydrocarbon group is substituted with a halogen atom. The halogenated hydrocarbon group may be a group in which all hydrogen atoms included in a hydrocarbon group are substituted with halogen atoms. Examples of the halogenated hydrocarbon group include halogenated alkyl groups and halogenated alkenyl groups.

Examples of the halogenated alkyl group include —CF₃, —CH₂F, —CH₂Br, —CH₂Cl, —CH₂I, and —CH₂CF₃. Examples of the halogenated alkenyl group include —CH═CHCF₃.

The oxygen-atom-containing substituent is a substituent having at least one selected from the group consisting of a hydroxy group, a carboxy group, an aldehyde group, an ether group, an acyl group, and an ester group.

Examples of the substituent having a hydroxy group include a hydroxy group itself and hydrocarbon groups having a hydroxy group. Regarding this substituent, the hydroxy group may be in the state of —O⁻ due to being deprotonated. Examples of the hydrocarbon group having a hydroxy group include —CH₂OH, —CH(OH)CH₃, —CH₂CH(OH)CH₃, and —CH₂C(OH)(CH₃)₂.

Examples of the substituent having a carboxy group include a carboxy group itself and hydrocarbon groups having a carboxy group. Regarding this substituent, the carboxy group may be in the state of —CO₂⁻ due to being deprotonated. Examples of the hydrocarbon group having a carboxy group include —CH₂CH₂COOH, —C(COOH)(CH₃)₂, and —CH₂CO₂.

Examples of the substituent having an aldehyde group include an aldehyde group itself and hydrocarbon groups having an aldehyde group. Examples of the hydrocarbon groups having an aldehyde group include —CH═CHCHO.

Examples of the substituent having an ether group include alkoxy groups, halogenated alkoxy groups, alkenyloxy groups, and oxiranyl groups and hydrocarbon groups having at least one of these functional groups. At least one hydrogen atom contained in the alkoxy group may be substituted with a group containing at least one atom selected from the group consisting of N, O, P, and S. Examples of the alkoxy group include a methoxy group, an ethoxy group, a 2-methoxyethoxy group, a butoxy group, a 2-methylbutoxy group, a 2-methoxybutoxy group, a 4-ethylthiobutoxy group, a pentyloxy group, a hexyloxy group, a heptyloxy group, an octyloxy group, a nonyloxy group, a decyloxy group, an undecyloxy group, a dodecyloxy group, a tridecyloxy group, a tetradecyloxy group, a pentadecyloxy group, a hexadecyloxy group, a heptadecyloxy group, an octadecyloxy group, a nonadecyloxy group, an eicosyloxy group, —OCH₂O⁻, —OCH₂CH₂O⁻, and —O(CH₂)₃O⁻. Examples of the halogenated alkoxy group include —OCHF₂, —OCH₂F, and —OCH₂Cl. Examples of the alkenyloxy group include —OCH═CH₂.

Examples of the hydrocarbon group having a functional group such as an alkoxy group include —CH₂OCH₃, —C(OCH₃)₃, a 2-methoxybutyl group, and a 6-methoxyhexyl group.

Examples of the substituent having an acyl group include an acyl group itself and hydrocarbon groups having an acyl group. Examples of the acyl group include —COCH₃. Examples of the hydrocarbon group having an acyl group include —CH═CHCOCH₃.

Examples of the substituent having an ester group include an alkoxycarbonyl group and an acyloxy group and hydrocarbon groups having at least one of these functional groups. Examples of the alkoxycarbonyl group include —COOCH₃, —COO(CH₂)₃CH₃, and —COO(CH₂)₇CH₃. Examples of the acyloxy group include —OCOCH₃. Examples of the hydrocarbon group having a functional group such as an acyloxy group include —CH₂OCOCH₃.

The nitrogen-atom-containing substituent is a substituent having, for example, at least one selected from the group consisting of an amino group, an imino group, a cyano group, an azi group, an amide group, a carbamate group, a nitro group, a cyanamide group, an isocyanate group, and an oxime group.

Examples of the substituent having an amino group include a primary amino groups, secondary amino groups, tertiary amino groups, and quaternary amino groups and hydrocarbon groups having at least one of these functional groups. Regarding this substituent, the amino group may be protonated. Examples of the tertiary amino group include —N(CH₃)₂. Examples of the hydrocarbon group having a functional group such as a primary amino group include —CH₂NH₂, —CH₂N(CH₃)₂, —(CH₂)₄N(CH₃)₂, —CH₂CH₂NH₃⁺, —CH₂CH₂NH(CH₃)₂⁺, and —CH₂CH₂N(CH₃)₃⁺.

Examples of the substituent having an imino group include an imino group itself and hydrocarbon groups having an imino group. Examples of the imino group include —N═CCl₂.

Examples of the substituent having a cyano group include a cyano group itself and hydrocarbon groups having a cyano group. Examples of the hydrocarbon group having a cyano group include —CH₂CN and —CH═CHCN.

Examples of the substituent having an azi group include an azi group itself and hydrocarbon groups having an azi group.

Examples of the substituent having an amide group include an amide group itself and hydrocarbon groups having an amide group. Examples of the amide group include —CONH₂, —NHCHO, —NHCOCH₃, —NHCOCF₃, —NHCOCH₂Cl, and —NHCOCH(CH₃)₂. Examples of the hydrocarbon group having an amide group include —CH₂CONH₂ and —CH₂NHCOCH₃.

Examples of the substituent having a carbamate group include a carbamate group itself and hydrocarbon groups having a carbamate group. Examples of the carbamate group include —NHCOOCH₃, —NHCOOCH₂CH₃, and —NHCO₂(CH₂)₃CH₃.

Examples of the substituent having a nitro group include a nitro group itself and hydrocarbon groups having a nitro group. Examples of the hydrocarbon group having a nitro group include —C(NO₂)(CH₃)₂.

Examples of the substituent having a cyanamide group include a cyanamide group itself and hydrocarbon groups having a cyanamide group. Examples of the hydrocarbon group having a cyanamide group include —NHCN.

Examples of the substituent having an isocyanate group include an isocyanate group itself and hydrocarbon groups having an isocyanate group. The isocyanate group is denoted by —N═C═O.

Examples of the substituent having an oxime group include an oxime group itself and hydrocarbon groups having an oxime group. The oxime group is denoted by —CH═NOH.

Examples of the sulfur-atom-containing substituent include substituents having at least one selected from the group consisting of a thiol group, a sulfide group, a sulfinyl group, a sulfonyl group, a sulfino group, a sulfonic acid group, an acylthio group, a sulfenamide group, a sulfonamide group, a thioamide group, a thiocarbamide group, and thiocyano group.

Examples of the substituent having a thiol group include a thiol group itself and hydrocarbon groups having a thiol group. The thiol group is denoted by —SH.

Examples of the substituent having a sulfide group include an alkylthio group, an alkyldithio group, an alkenylthio group, an alkynylthio group, and a thiacyclopropyl group and hydrocarbon groups having at least one of these functional groups. At least one hydrogen atom contained in the alkylthio group may be substituted with a halogen group. Examples of the alkylthio group include —SCH₃, —S(CH₂)F, —SCH(CH₃)₂, and —SCH₂CH₃. Examples of the alkyldithio group include —SSCH₃. Examples of the alkenylthio group include —SCH═CH₂ and —SCH₂CH═CH₂. Examples of the alkenylthio group include —SC≡CH. Examples of the hydrocarbon group having a functional group such as an alkylthio group include —CH₂SCF₃.

Examples of the substituent having a sulfinyl group include a sulfinyl group itself and hydrocarbon groups having a sulfinyl group. Examples of the sulfinyl group include —SOCH₃.

Examples of the substituent having a sulfonyl group include a sulfonyl group itself and hydrocarbon groups having a sulfonyl group. Examples of the sulfonyl group include —SO₂CH₃. Examples of the hydrocarbon group having a sulfonyl group include —CH₂SO₂CH₃ and —CH₂SO₂CH₂CH₃.

Examples of the substituent having a sulfino group include a sulfino group itself and hydrocarbon groups having a sulfino group. Regarding this substituent, the sulfino group may be in the state of —SO₂⁻ due to being deprotonated.

Examples of the substituent having a sulfonic acid group include a sulfonic acid group itself and hydrocarbon groups having a sulfonic acid group. Regarding this substituent, the sulfonic acid group may be in the state of —SO₃⁻ due to being deprotonated.

Examples of the substituent having an acylthio group include an acylthio group itself and hydrocarbon groups having an acylthio group. Examples of the acylthio group include —SCOCH₃.

Examples of the substituent having a sulfenamido group include a sulfenamido group itself and hydrocarbon groups having a sulfenamido group. Examples of the sulfenamido group include —SN(CH₃)₂.

Examples of the substituent having a sulfonamide group include a sulfonamide group itself and hydrocarbon groups having a sulfonamide group. Examples of the sulfonamide group include —SO₂NH₂ and —NHSO₂CH₃.

Examples of the substituent having a thioamide group include a thioamide group itself and hydrocarbon groups having a thioamide group. Examples of the thioamide group include —NHCSCH₃. Examples of the hydrocarbon group having a thioamide group include —CH₂SC(NH₂)₂⁺.

Examples of the substituent having a thiocarbamide group include a thiocarbamide group itself and hydrocarbon groups having a thiocarbamide group.

Examples of the thiocarbamide group include —NHCSNHCH₂CH₃.

Examples of the substituent having a thiocyano group include a thiocyano group itself and hydrocarbon groups having a thiocyano group. Examples of the hydrocarbon group having a thiocyano group include —CH₂SCN.

Examples of the silicon-atom-containing substituent include substituents having at least one selected from the group consisting of a silyl group and a siloxy group.

Examples of the substituent having a silyl group include a silyl group itself and hydrocarbon groups having a silyl group. Examples of the silyl group include —Si(CH₃)₃, —SiH(CH₃)₂, —Si(OCH₃)₃, —Si(OCH₂CH₃)₃, —SiCH₃(OCH₃)₂, —Si(CH₃)₂OCH₃, —Si(N(CH₃)₂)₃, —SiF(CH₃)₂, —Si(OSi(CH₃)₃)₃, and —Si(CH₃)₂OSi(CH₃)₃). Examples of the hydrocarbon group having a silyl group include —(CH₂)₂Si(CH₃)₃.

Examples of the substituent having a siloxy group include a siloxy group itself and hydrocarbon groups having a siloxy group. Examples of the hydrocarbon group having a siloxy group include —CH₂OSi(CH₃)₃.

Examples of the phosphorus-atom-containing substituent include substituents having at least one selected from the group consisting of a phosphino group and a phosphoryl group.

Examples of the substituent having a phosphino group include a phosphino group itself and hydrocarbon groups having a phosphino group. Examples of the phosphino group include —PH₂, —P(CH₃)₂, —P(CH₂CH₃)₂, —P(C(CH₃)₃)₂, and —P(CH(CH₃)₂)₂.

Examples of the substituent having a phosphoryl group include a phosphoryl group itself and hydrocarbon groups having a phosphoryl group. Examples of the hydrocarbon group having a phosphoryl group include —CH₂PO(OCH₂CH₃)₂.

Examples of the boron-atom-containing substituent include substituents having a boronic acid group. Examples of the hydrocarbon group having a boronic acid group include a boronic acid group itself and hydrocarbon groups having a boronic acid group.

In this regard, Compound A satisfies the following requirements (a) and (b).
(a) R¹, R², R⁶, R⁷, R¹², R¹⁷, and R²² each represent a substituent other than a substituent having an aromatic ring.
(b) R¹ to R²² are each independently a hydrogen atom or a substituent having Hammett substituent constant σₚ within the range of greater than or equal to −0.2 and less than or equal to 0.2.

Regarding the requirement (a), R¹, R², R⁶, R⁷, R¹², R¹⁷, and R²² do not have an aromatic ring. The aromatic rings include not only aromatic rings composed of only carbon atoms but also heteroaromatic rings containing a hetero atom, such as an oxygen atom, a nitrogen atom, or a sulfur atom. Examples of the aromatic ring include a benzene ring, a naphthalene ring, an anthracene ring, a phenanthrene ring, a furane ring, a pyrrole ring, a pyridine ring, and a thiophene ring. Specific examples of the substituent having an aromatic ring include an aryl group (—Ar) and an arylethynyl group (—C≡C—Ar). R¹ to R²² may be each a substituent other than a substituent having an aromatic ring.

Regarding the requirement (b), Hammett substituent constant σₚ is an indicator of the electron-withdrawing property and the electron-donating property of the substituent. The substituent constants σₚ of individual substituents are disclosed in, for example, CORWIN HANSCH et al, “A Survey of Hammett Substituent Constants and Resonance and Field Parameters”, Chem. Rev. 1991, Vol. 91, p. 165-195. Examples of the substituent having a substituent constant σₚ within the range of greater than or equal to −0.2 and less than or equal to 0.2 include —F, —I, hydrocarbon groups, and a silyl group. When at least two of R¹ to R²² are substituents other than a hydrogen atom, each of the substituents may be independently —F, —CH₃, —CH₂CH₂CH₃, or —Si(CH₃)₃.

The substituent having a substituent constant σₚ within the range of greater than or equal to −0.2 and less than or equal to 0.2 tends to have a low electron-withdrawing property or a low electron-donating property. Therefore, regarding Compound A satisfying the requirement (b), an increase in the energy of the HOMO and a decrease in the energy of the LUMO due to the electron-withdrawing property or the electron-donating property of the substituent can be suppressed from occurring. That is, the energy gap between the HOMO and the LUMO can be suppressed from decreasing. Consequently, the peak due to single-photon absorption can be suppressed from shifting to a long wavelength, and the ratio σ/ε with respect to the light having a wavelength in a short wavelength range can be suppressed from decreasing. Compound A satisfying the requirement (b) tends to have high non-linearity of light absorption with respect to the light having a wavelength in a short wavelength range.

In Compound A, R¹ to R²² may each represent a hydrogen atom. In such an instance, Compound A tends to have high non-linearity of light absorption with respect to the light having a wavelength in a short wavelength range.

In Formula (1), L¹ and L² each independently represent a single bond or —C≡C—. L¹ and L² may be the same or differ from each other. As an example, each of L¹ and L² may represents a single bond. In particular, Compound A may be Compound B denoted by Formula (2) below.

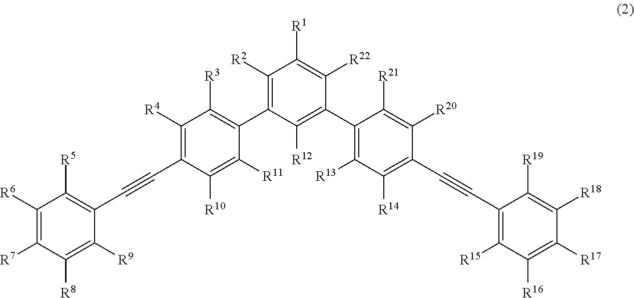

R¹ to R²² in Formula (2) are the same as that described above in Formula (1). Specific examples of combinations of R¹ to R²² in Formula (2) are presented in Table 1 to Table 3 below. In Table 1 to Table 3, the column “Compound” indicates the abbreviated designation of Compound B containing specific R¹ to R²². Me represents —CH₃. Pr represents —CH₂CH₂CH₃.

TABLE 1

| | | | | | | | | R⁸ to | | R¹³ to |
|---|---|---|---|---|---|---|---|---|---|---|
| Compound | R¹ | R² | R³ | R⁴ | R⁵ | R⁶ | R⁷ | R¹¹ | R¹² | R²² |
| (2)-1 | H | H | H | H | H | H | H | H | H | H |
| (2)-2 | CH₃ | H | H | H | H | H | H | H | H | H |
| (2)-3 | H | CH₃ | H | H | H | H | H | H | H | H |
| (2)-4 | H | H | CH₃ | H | H | H | H | H | H | H |
| (2)-5 | H | H | H | CH₃ | H | H | H | H | H | H |
| (2)-6 | H | H | H | H | CH₃ | H | H | H | H | H |
| (2)-7 | H | H | H | H | H | CH₃ | H | H | H | H |
| (2)-8 | H | H | H | H | H | H | CH₃ | H | H | H |
| (2)-9 | H | H | H | H | H | H | H | H | CH₃ | H |
| (2)-10 | Pr | H | H | H | H | H | H | H | H | H |
| (2)-11 | H | Pr | H | H | H | H | H | H | H | H |
| (2)-12 | H | H | Pr | H | H | H | H | H | H | H |
| (2)-13 | H | H | H | Pr | H | H | H | H | H | H |
| (2)-14 | H | H | H | H | Pr | H | H | H | H | H |
| (2)-15 | H | H | H | H | H | Pr | H | H | H | H |
| (2)-16 | H | H | H | H | H | H | Pr | H | H | H |
| (2)-17 | H | H | H | H | H | H | H | H | Pr | H |
| (2)-18 | F | H | H | H | H | H | H | H | H | H |
| (2)-19 | H | F | H | H | H | H | H | H | H | H |
| (2)-20 | H | H | F | H | H | H | H | H | H | H |
| (2)-21 | H | H | H | F | H | H | H | H | H | H |
| (2)-22 | H | H | H | H | F | H | H | H | H | H |
| (2)-23 | H | H | H | H | H | F | H | H | H | H |
| (2)-24 | H | H | H | H | H | H | F | H | H | H |
| (2)-25 | H | H | H | H | H | H | H | H | F | H |
| (2)-26 | SiMe₃ | H | H | H | H | H | H | H | H | H |
| (2)-27 | H | SiMe₃ | H | H | H | H | H | H | H | H |
| (2)-28 | H | H | SiMe₃ | H | H | H | H | H | H | H |
| (2)-29 | H | H | H | SiMe₃ | H | H | H | H | H | H |
| (2)-30 | H | H | H | H | SiMe₃ | H | H | H | H | H |

TABLE 2

| | | | | | | | | R⁸ to | | R¹³ to |
|---|---|---|---|---|---|---|---|---|---|---|
| Compound | R¹ | R² | R³ | R⁴ | R⁵ | R⁶ | R⁷ | R¹¹ | R¹² | R²² |
| (2)-31 | H | H | H | H | H | SiMe₃ | H | H | H | H |
| (2)-32 | H | H | H | H | H | H | SiMe₃ | H | H | H |
| (2)-33 | H | H | H | H | H | H | H | H | SiMe₃ | H |

TABLE 3

| Compound | R¹ to R⁶ | R⁷ | R⁸ to R¹⁶ | R¹⁷ | R¹⁸ to R²² |
|---|---|---|---|---|---|
| (2)-34 | H | CH₃ | H | CH₃ | H |
| (2)-35 | H | Pr | H | Pr | H |
| (2)-36 | H | F | H | F | H |
| (2)-37 | H | SiMe₃ | H | SiMe₃ | H |

In Formula (1), L¹ and L² may each represent —C≡C—. In particular, Compound A may be Compound C denoted by Formula (3) below.

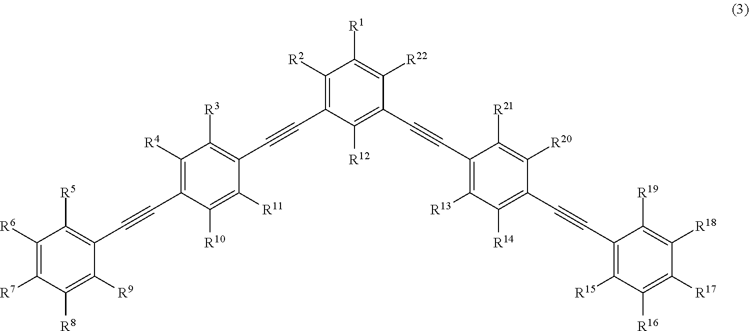

R¹ to R²² in Formula (3) are the same as that described above in Formula (1). Specific examples of combinations of R¹ to R²² in Formula (3) are presented in Table 4 to Table 6 below. In Table 4 to Table 6, the column “Compound” indicates the abbreviated designation of Compound C containing specific R¹ to R²².

TABLE 4

| | | | | | | | | R⁸ to | | R¹³ to |
|---|---|---|---|---|---|---|---|---|---|---|
| Compound | R¹ | R² | R³ | R⁴ | R⁵ | R⁶ | R⁷ | R¹¹ | R¹² | R²² |
| (3)-1 | H | H | H | H | H | H | H | H | H | H |
| (3)-2 | CH₃ | H | H | H | H | H | H | H | H | H |
| (3)-3 | H | CH₃ | H | H | H | H | H | H | H | H |
| (3)-4 | H | H | CH₃ | H | H | H | H | H | H | H |
| (3)-5 | H | H | H | CH₃ | H | H | H | H | H | H |
| (3)-6 | H | H | H | H | CH₃ | H | H | H | H | H |
| (3)-7 | H | H | H | H | H | CH₃ | H | H | H | H |
| (3)-8 | H | H | H | H | H | H | CH₃ | H | H | H |
| (3)-9 | H | H | H | H | H | H | H | H | CH₃ | H |
| (3)-10 | Pr | H | H | H | H | H | H | H | H | H |
| (3)-11 | H | Pr | H | H | H | H | H | H | H | H |
| (3)-12 | H | H | Pr | H | H | H | H | H | H | H |
| (3)-13 | H | H | H | Pr | H | H | H | H | H | H |
| (3)-14 | H | H | H | H | Pr | H | H | H | H | H |
| (3)-15 | H | H | H | H | H | Pr | H | H | H | H |
| (3)-16 | H | H | H | H | H | H | Pr | H | H | H |
| (3)-17 | H | H | H | H | H | H | H | H | Pr | H |
| (3)-18 | F | H | H | H | H | H | H | H | H | H |
| (3)-19 | H | F | H | H | H | H | H | H | H | H |
| (3)-20 | H | H | F | H | H | H | H | H | H | H |
| (3)-21 | H | H | H | F | H | H | H | H | H | H |
| (3)-22 | H | H | H | H | F | H | H | H | H | H |
| (3)-23 | H | H | H | H | H | F | H | H | H | H |
| (3)-24 | H | H | H | H | H | H | F | H | H | H |
| (3)-25 | H | H | H | H | H | H | H | H | F | H |
| (3)-26 | SiMe₃ | H | H | H | H | H | H | H | H | H |
| (3)-27 | H | SiMe₃ | H | H | H | H | H | H | H | H |
| (3)-28 | H | H | SiMe₃ | H | H | H | H | H | H | H |
| (3)-29 | H | H | H | SiMe₃ | H | H | H | H | H | H |
| (3)-30 | H | H | H | H | SiMe₃ | H | H | H | H | H |

TABLE 5

| | | | | | | | | R⁸ to | | R¹³ to |
|---|---|---|---|---|---|---|---|---|---|---|
| Compound | R¹ | R² | R³ | R⁴ | R⁵ | R⁶ | R⁷ | R¹¹ | R¹² | R²² |
| (3)-31 | H | H | H | H | H | SiMe₃ | H | H | H | H |
| (3)-32 | H | H | H | H | H | H | SiMe₃ | H | H | H |
| (3)-33 | H | H | H | H | H | H | H | H | SiMe₃ | H |

TABLE 6

| Compound | R¹ to R⁶ | R⁷ | R⁸ to R¹⁶ | R¹⁷ | R¹⁸ to R²² |
|---|---|---|---|---|---|
| (3)-34 | H | CH₃ | H | CH₃ | H |
| (3)-35 | H | Pr | H | Pr | H |
| (3)-36 | H | F | H | F | H |
| (3)-37 | H | SiMe₃ | H | SiMe₃ | H |

There is no particular limitation regarding a method for synthesizing Compound B denoted by Formula (2) and Compound C denoted by Formula (3), and, for example, the Sonogashira coupling reaction or the like can be used. Compound B denoted by Formula (2) can be synthesized by, for example, the following method. Initially, Compound D denoted by Formula (4) below, Compound E denoted by Formula (5) below, and Compound F denoted by Formula (6) below are prepared.

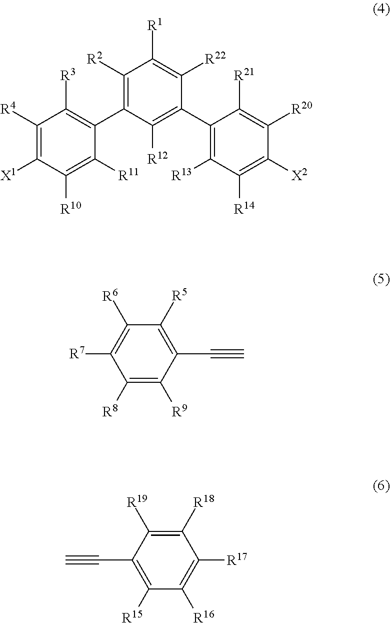

In Formula (4), X¹ and X² are each independently a halogen atom or B(OH)₂. Examples of the halogen atom in X¹ and X² include Br and I. R¹ to R⁴, R¹⁰ to R¹⁴, and R²⁰ to R²² in Formula (4) are the same as that described above in Formula (1). R⁵ to R⁹ in Formula (5) and R¹⁵ to R¹⁹ in Formula (6) are also the same as that described above in Formula (1).

Subsequently, a coupling reaction of Compound D, Compound E, and Compound F is performed. Consequently, Compound B can be synthesized. The condition of the coupling reaction can be appropriately adjusted in accordance with, for example, types of the substituents contained in Compound D, Compound E, and Compound F.

Compound C denoted by Formula (3) can be synthesized by, for example, the following method. Initially, Compound G denoted by Formula (7) below, Compound H denoted by Formula (8) below, and Compound I denoted by Formula (9) below are prepared.

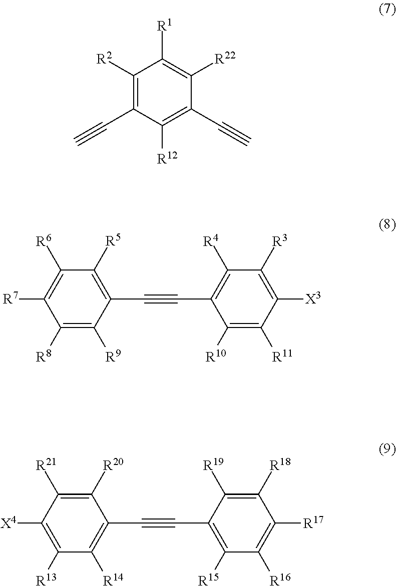

X³ in Formula (8) and X⁴ in Formula (9) are each independently a halogen atom or B(OH)₂. Examples of the halogen atom in X³ and X⁴ include Br and I. R¹, R², R¹², and R²² in Formula (7) are the same as that described above in Formula (1). R³ to R¹¹ in Formula (8) and R¹³ to R²¹ in Formula (9) are also the same as that described above in Formula (1).

Subsequently, a coupling reaction of Compound G, Compound H, and Compound I is performed. Consequently, Compound C can be synthesized. The condition of the coupling reaction can be appropriately adjusted in accordance with, for example, types of the substituents contained in Compound G, Compound H, and Compound I.

The Compound A denoted by Formula (1) tends to have excellent two-photon absorption characteristics and have low single-photon absorption with respect to the light having a wavelength in a short wavelength range. As an example, when Compound A is irradiated with the light having a wavelength of 405 nm, in Compound A, two-photon absorption occurs, whereas substantially no single-photon absorption may occur.

The two-photon absorption cross-sectional area of Compound A with respect to the light having a wavelength of 405 nm may be greater than 1 GM, may be greater than or equal to 10 GM, may be greater than or equal to 20 GM, may be greater than or equal to 100 GM, may be greater than or equal to 400 GM, or may be greater than or equal to 600 GM. There is no particular limitation regarding the upper limit value of the two-photon absorption cross-sectional area of Compound A, and the upper limit value is, for example, 10,000 GM or may be 1,000 GM. The two-photon absorption cross-sectional area can be measured by, for example, the Z-scan method described in J. Opt. Soc. Am. B, 2003, Vol. 20, p. 529. The Z-scan method is widely used as a method for measuring a non-linear optical constant. Regarding the Z-scan method, around the focal point at which the laser beam is condensed, a measurement sample is moved in the beam irradiation direction. In such an instance, a change in the amount of the light that passes through the measurement sample is recorded. Regarding the Z-scan method, the power density of the incident light changes in accordance with the position of the measurement sample. Therefore, when the measurement sample performs non-linear optical absorption, the measurement sample being positioned around the focal point of the laser beam attenuates the amount of the light that is passed. The two-photon absorption cross-sectional area can be calculated by fitting a change in the amount of the light that is passed to the theoretical curve predicted based on the intensity of the incident light, the thickness of the measurement sample, the concentration of Compound A in the measurement sample, and the like.

The two-photon absorption cross-sectional area may be a calculation value by using the computational chemistry. Some methods for estimating the two-photon absorption cross-sectional area by using the computational chemistry have been proposed. The calculation value of the two-photon absorption cross-sectional area can be determined in accordance with the second-order non-linear response theory described in, for example, J. Chem. Theory Comput. 2018, Vol. 14, p. 807.

The molar extinction coefficient of Compound A with respect to the light having a wavelength of 405 nm may be less than or equal to 100 mol⁻¹·L·cm⁻¹, may be less than or equal to 10 mol⁻¹·L·cm⁻¹, may be less than or equal to 1 mol⁻¹·L·cm⁻¹, or may be less than or equal to 0.1 mol¹·L·cm⁻¹. There is no particular limitation regarding the lower limit value of the molar extinction coefficient of Compound A, and the lower limit value is, for example, 0.00001 mol⁻¹·L·cm⁻¹. The molar extinction coefficient can be measured by a method in conformity with the specification of Japanese Industrial Standards (JIS) K 0115:2004. In the measurement of the molar extinction coefficient, a light source which applies light having a photon density at which almost no two-photon absorption by Compound A occurs is used. Further, in the measurement of the molar extinction coefficient, the concentration of the Compound A is adjusted to 500 mmol/L. This concentration is very high value compared with the concentration in a test for measuring the molar extinction coefficient at a light absorption peak. The molar extinction coefficient can be utilized as an indicator of single-photon absorption.

The molar extinction coefficient may be a calculation value by using a quantum chemistry calculation program. Regarding the quantum chemistry calculation program, for example, Gaussian 16 (produced by Gaussian) can be used.

Compound A tends to have a large ratio σ/ε of the two-photon absorption cross-sectional area σ (GM) to the molar extinction coefficient ε (mol¹·L·cm⁻¹) with respect to the light having a wavelength in a short wavelength range. The ratio σ/ε of Compound A with respect to the light having a wavelength of 405 nm may be greater than or equal to 20, may be greater than or equal to 50, may be greater than or equal to 100, may be greater than or equal to 500, may be greater than or equal to 1,000, may be greater than or equal to 1,500, or may be greater than or equal to 2,000. There is no particular limitation regarding the upper limit value of the ratio σ/ε of Compound A, and the upper limit value is, for example, 50,000 and may be 20,000.

When Compound A performs two-photon absorption, Compound A absorbs about twice as much energy as the light applied to Compound A. The wavelength of the light having about twice as much energy as the light having a wavelength of 405 nm is, for example, 200 nm. When the light having a wavelength around 200 nm is applied to Compound A, single-photon absorption may occur in Compound A. Further, in Compound A, single-photon absorption may occur with respect to the light having a wavelength around the wavelength range in which two-photon absorption occurs.

Compound A also tends to emit fluorescence. The wavelength of the fluorescence emitted from Compound A may be greater than or equal to 405 nm and less than or equal to 660 nm and may be sometimes greater than or equal to 300 nm and less than or equal to 650 nm. The quantum yield Φf of the fluorescence of Compound A may be greater than or equal to 0.05, may be greater than or equal to 0.1, or may be greater than or equal to 0.5. There is no particular limitation regarding the upper limit value of the quantum yield Φf of the fluorescence of Compound A, and the upper limit value is, for example, 0.99. In the present specification, “quantum yield” means an internal quantum yield. The quantum yield of the fluorescence can be measured by using a commercially available absolute PL quantum yield spectrometer.

Compound A denoted by Formula (1) can be used as, for example, a component of an optical absorbing material. That is, the present disclosure provides an optical absorbing material containing Compound A denoted by Formula (1) from the viewpoint of such another aspect. The optical absorbing material contains, for example, Compound A as a primary component. Here, “primary component” means a component contained in the optical absorbing material with the largest content on a weight ratio basis. The optical absorbing material consists essentially of, for example, Compound A. Here, “consists essentially of” means to exclude other components that change essential features of the material concerned. However, the optical absorbing material may contain impurities in addition to Compound A.

The optical absorbing material functions as, for example, a non-linear optical absorbing material such as a two-photon-absorbing material. In particular, the optical absorbing material containing Compound A has excellent two-photon absorption characteristics with respect to the light having a wavelength in a short wavelength range. The present disclosure provides a non-linear optical absorbing material containing Compound A denoted by Formula (1) from the viewpoint of such another aspect.

Compound A is used for, for example, a device that utilizes the light having a wavelength in a short wavelength range. As an example, Compound A is used for a device that utilizes the light having a wavelength of greater than or equal to 390 nm and less than or equal to 420 nm. Examples of such a device include recording mediums, shaping machines, and fluorescent microscopes. Examples of the recording medium include three-dimensional optical memories. Specific examples of the three-dimensional optical memory include three-dimensional optical discs. Examples of the shaping machine include three-dimensional (3D) laser microfabrication machines, such as 3D printers. Examples of the fluorescent microscope include two-photon fluorescent microscopes. The light utilized for these devices have, for example, a high photon density around the focal point of the light. The power density around the focal point of the light utilized for the device is, for example, greater than or equal to 0.1 W/cm² and less than or equal to 1.0×10²⁰ W/cm². The power density around the focal point of the light may be greater than or equal to 1.0 W/cm², may be greater than or equal to 1.0×10² W/cm², or may be greater than or equal to 1.0×10⁵ W/cm². Regarding the light source of the device, for example, a femtosecond laser, such as a titanium-sapphire laser, or a pulse laser having a pulse width of picoseconds to nanoseconds, such as a semiconductor laser, can be used.

The recording medium includes, for example, a thin film referred to as a recording layer. Regarding the recording medium, information is recorded in the recording layer. As an example, a thin film serving as the recording layer contains Compound A. That is, the present disclosure provides the recording medium containing Compound A above from the viewpoint of such another aspect.

The recording layer may further contain, in addition to Compound A, a polymer compound that functions as a binder. The recording medium may include, in addition to the recording layer, a dielectric layer. The recording medium includes, for example, a plurality of recording layers and a plurality of dielectric layers. In the recording medium, the plurality of recording layers and the plurality of dielectric layers may be stacked alternately.

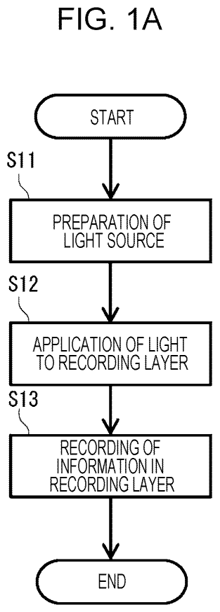

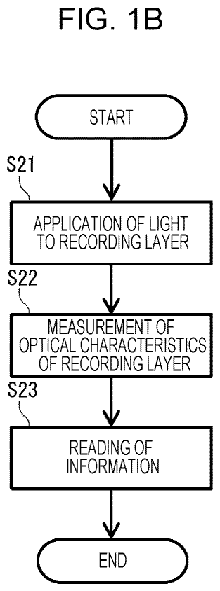

Next, an information recording method by using the above-described recording medium will be described. FIG. 1A is a flow chart illustrating the information recording method by using the above-described recording medium. Initially, in Step S11, a light source that emits light having a wavelength of greater than or equal to 390 nm and less than or equal to 420 nm is prepared. Regarding the light source, for example, a femtosecond laser, such as a titanium-sapphire laser, or a pulse laser having a pulse width of picoseconds to nanoseconds, such as a semiconductor laser, can be used. Subsequently, in Step S12, the light from the light source is condensed using a lens or the like and is applied to the recording layer of the recording medium. In particular, the light from the light source is condensed using a lens or the like and is applied to the recording region in the recording layer of the recording medium. The power density around the focal point of the light is, for example, greater than or equal to 0.1 W/cm² and less than or equal to 1.0×10²⁰ W/cm². The power density around the focal point of the light may be greater than or equal to 1.0 W/cm², may be greater than or equal to 1.0×10² W/cm², or may be greater than or equal to 1.0×10⁵ W/cm². In the present specification, “recording region” means a spot that is present in the recording layer and that can record information by being irradiated with the light.

In the recording region irradiated with the above-described light, a physical change or a chemical change occurs, and the optical characteristics of the recording region changes. For example, the intensity of the fluorescent light emitted from the recording region is lowered. Regarding the recording region irradiated with the light, the intensity of the light reflected in the recording region, the reflectance of the light in the recording region, the absorptance of the light in the recording region, the refractive index of the light in the recording region, and the wavelength of the fluorescence emitted from the recording region may change. Consequently, the information can be recorded in the recording layer, in particular, in the recording region (Step S13).

Next, an information reading method by using the above-described recording medium will be described. FIG. 1B is a flow chart illustrating the information reading method by using the above-described recording medium. Initially, in Step S21, the light is applied to the recording layer of the recording medium. In particular, the light is applied to the recording region in the recording layer of the recording medium. The light used in Step S21 may be the same as or differ from the light utilized to record the information in the recording medium. Subsequently, in Step S22, the optical characteristics of the recording layer are measured. In particular, the optical characteristics of the recording region are measured. In Step S22, for example, the intensity of the fluorescence emitted from the recording region is measured. In Step S22, regarding the optical characteristics of the recording region, the intensity of the light reflected in the recording region, the reflectance of the light in the recording region, the absorptance of the light in the recording region, the refractive index of the light in the recording region, and the wavelength of the fluorescence emitted from the recording region may be measured. Thereafter, in Step S23, the information is read from the recording layer, in particular, from the recording region.

In the information reading method, the recording region in which information was recorded can be searched by the following method. Initially, light is applied to a specific region of the recording medium. The light may be the same as or differ from the light utilized to record the information in the recording medium. Subsequently, the optical characteristics of the region irradiated with the light are measured. Examples of the optical characteristics include the intensity of the fluorescence emitted from the region, the intensity of the light reflected in the region, the reflectance of the light in the region, the absorptance of the light in the region, the refractive index of the light in the region, and the wavelength of the fluorescence emitted from the region. Whether the region irradiated with the light is the recording region is determined in accordance with the measured optical characteristics. For example, when the intensity of the fluorescence emitted from the region is less than or equal to a specific value, it is determined that the region is the recording region. On the other hand, when the intensity of the fluorescence is greater than the specific value, it is determined that the region is not the recording region. In this regard, the method for determining whether the region irradiated with the light is the recording region is not limited to the above-described method. For example, when the intensity of the fluorescence emitted from the region is greater than the specific value, it may be determined that the region is the recording region. In addition, when the intensity of the fluorescence emitted from the region is less than or equal to the specific value, it may be determined that the region is not the recording region. When it is determined that the region is not the recording region, the same operation is performed with respect to another region of the recording medium. Consequently, the recording region can be searched.

The information recording method and the information reading method by using the above-described recording medium can be performed by using, for example, a known recording apparatus. The recording apparatus includes, for example, a light source for applying light to the recording region of the recording medium, a measuring instrument for measuring the optical characteristics of the recording region, and a controller for controlling the light source and the measuring instrument.

A shaping machine performs shaping by, for example, applying light to a photo-curable resin composition so as to cure the resin composition. As an example, the photo-curable resin composition for three-dimensional (3D) laser microfabrication contains Compound A. The photo-curable resin composition contains, in addition to Compound A, a polymerizable compound and a polymerization initiator, for example. The photo-curable resin composition may further contain additives, such as a binder resin. The photo-curable resin composition may contain an epoxy resin.

According to a fluorescent microscope, for example, light is applied to a living body sample containing a fluorochrome material, and the fluorescence emitted from the fluorochrome material can be observed. As an example, the fluorochrome material to be added to the living body contains Compound A.

### EXAMPLE

The present disclosure will be described below in further detail with reference to the examples. In this regard, the following examples are exemplifications, and the present disclosure is not limited to the following examples.

### Synthesis of Compound (2)-1

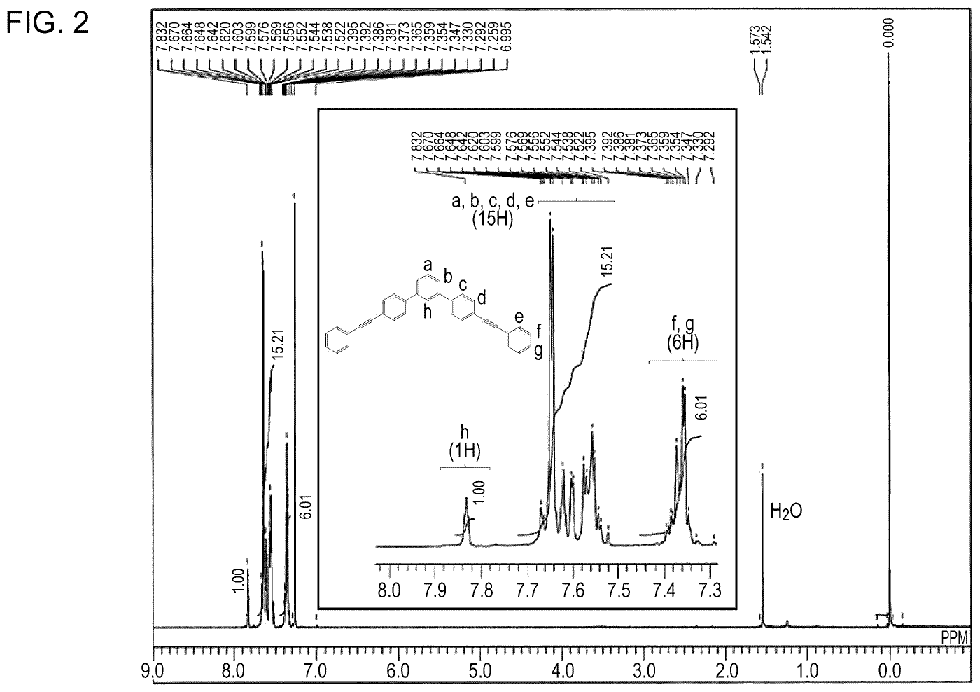

A reaction vessel having a volume of 50 mL was charged with 2.0 g (5.1 mmol) of 4,4″-dibromo-1,1′:3′,1″-terphenyl (produced by TOKYO KASEI KOGYO CO., LTD.), 0.03 g (0.15 mmol) of copper(I) iodide (produced by FUJIFILM Wako Pure Chemical Corporation), 20 mL of tetrahydrofuran (produced by FUJIFILM Wako Pure Chemical Corporation), and 10 mL of diisopropylamine (produced by TOKYO KASEI KOGYO CO., LTD.). The interior of the reaction vessel was subjected to deaeration treatment, and replacement with an argon gas was further performed. Thereafter, 2.1 mL (20.6 mmol) of phenylacetylene (produced by TOKYO KASEI KOGYO CO., LTD.), 1.0 mL (0.52 mmol) of solution containing tri-tert-butylphosphine (produced by TOKYO KASEI KOGYO CO., LTD.) at a concentration of 0.5 mol/L, and 0.03 g (0.15 mmol) of palladium(II) acetate (TOKYO KASEI KOGYO CO., LTD.) were added to the solution in the reaction vessel. An oil bath was used, and the solution was heat-agitated at an internal temperature of 40° C. for 3 hours. The solution was naturally cooled, and 40 mL of methanol (produced by FUJIFILM Wako Pure Chemical Corporation) was added so as to precipitate a solid. The resulting solid was subjected to filtration and recrystallization in toluene (produced by FUJIFILM Wako Pure Chemical Corporation) so as to synthesize Compound (2)-1 in Table 1 above. Compound (2)-1 was identified by using ¹H-NMR. FIG. 2 is a graph illustrating a ¹H-NMR spectrum of Compound (2)-1. The ¹H-NMR spectrum of Compound (2)-1 was as described below.

¹H-NMR (600 MHz, CHLOROFORM-D) δ 7.83 (s, 1H), 7.67-7.52 (m, 15H), 7.40-7.35 (m, 6H)

### Synthesis of Compound (3)-1

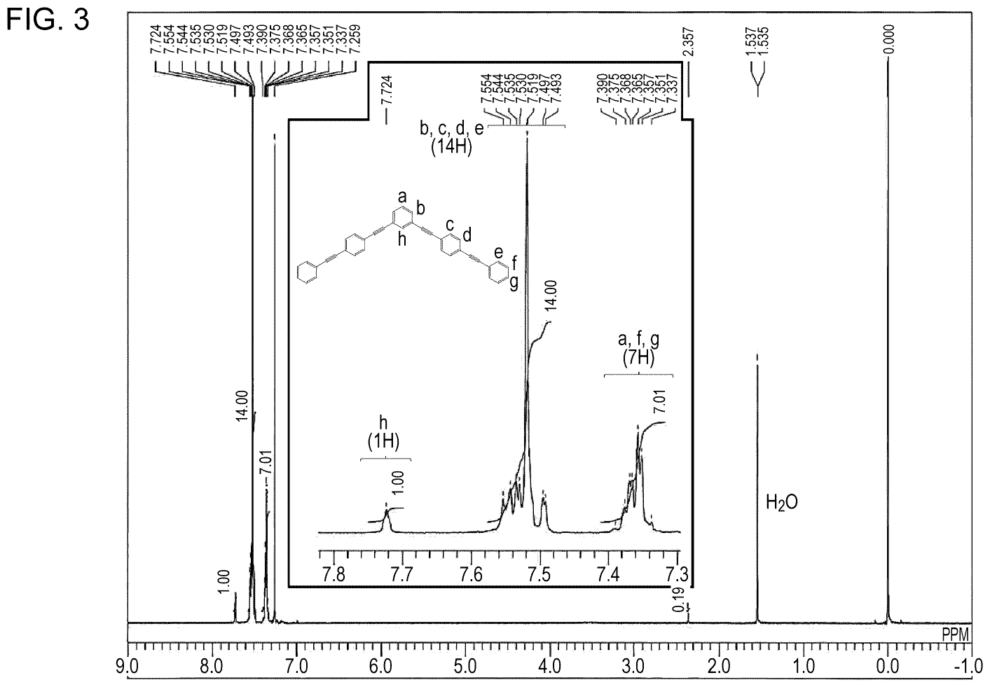

A reaction vessel having a volume of 30 mL was charged with 4.5 g (17.4 mmol) of 1-bromo-4-phenylethynylbenzene (produced by TOKYO KASEI KOGYO CO., LTD.), 0.04 g (0.16 mmol) of copper(I) iodide (produced by FUJIFILM Wako Pure Chemical Corporation), 10 mL of tetrahydrofuran (produced by FUJIFILM Wako Pure Chemical Corporation), and 5.0 mL diisopropylamine (produced by TOKYO KASEI KOGYO CO., LTD.), and deaeration treatment was performed for 10 min. Thereafter, 1.0 g (7.9 mmol) of 1,3-diethynylbenzene (produced by TOKYO KASEI KOGYO CO., LTD.), 3.0 mL (1.58 mmol) of solution containing tri-tert-butylphosphine (produced by TOKYO KASEI KOGYO CO., LTD.) at a concentration of 0.5 mol/L, and 0.2 g (0.23 mmol) of bis(dibenzylideneaceton)palladium(0) (TOKYO KASEI KOGYO CO., LTD.) were added to the solution in the reaction vessel. An oil bath was used, and the solution was heat-agitated at an internal temperature of 50° C. for 12 hours. The resulting suspension liquid was naturally cooled to room temperature, city water was added, and extraction was performed using toluene. The extract was washed with saturated saline solution, and drying treatment was performed using anhydrous magnesium sulfate. Subsequently, the extract was concentrated so as to obtain a brown solid. The brown solid was washed with methanol and refined by silica gel column chromatography so as to synthesize Compound (3)-1 in Table 4 above. Compound (3)-1 was identified by using ¹H-NMR. FIG. 3 is a graph illustrating a ¹H-NMR spectrum of Compound (3)-1. The ¹H-NMR spectrum of Compound (3)-1 was as described below.

¹H-NMR (600 MHz, CHLOROFORM-D) δ 7.72 (s, 1H), 7.55-7.49 (m, 14H), 7.39-7.34 (m, 7H)

### Compounds of Comparative Examples 1 to 8

The compounds of Comparative examples 2 to 5 presented in Table 11 below were prepared. The compounds of Comparative examples 1 to 8 are denoted by Formula (10) to Formula (17) below, respectively. In this regard, of Compounds A denoted by Formula (1), the compound denoted by Formula (10) corresponds to a compound that does not satisfy the requirement (a). Of Compounds A denoted by Formula (1), the compounds denoted by Formula (15) to Formula (17) correspond to compounds that do not satisfy the requirement (b). Regarding Compound D-29 denoted by Formula (11) below, that was the compound of Comparative example 2, the compound synthesized in conformity with the method described in paragraphs [0222] to [0230] of Japanese Patent No. 5659189 was used. Regarding Compound if denoted by Formula (12) below, that was the compound of Comparative example 3, the compound synthesized in conformity with the method described in a paragraph [0083] of Japanese Patent No. 5821661 was used. Regarding hexakis(phenylethynyl)benzene (HPEB) denoted by Formula (13) below, that was the compound of Comparative example 4, the compound synthesized in conformity with the method described in K. Kondo et al., j. Chem. Soc., Chem. Commun. 1995, 55-56 and W. Tao et al., J. Org. Chem. 1990, 55, 63-66 was used. Regarding 1,3,5-tri(phenylethynyl)benzene denoted by Formula (14) below, that was the compound of Comparative example 5, the compound produced by Sigma-Aldrich was purchased and used.

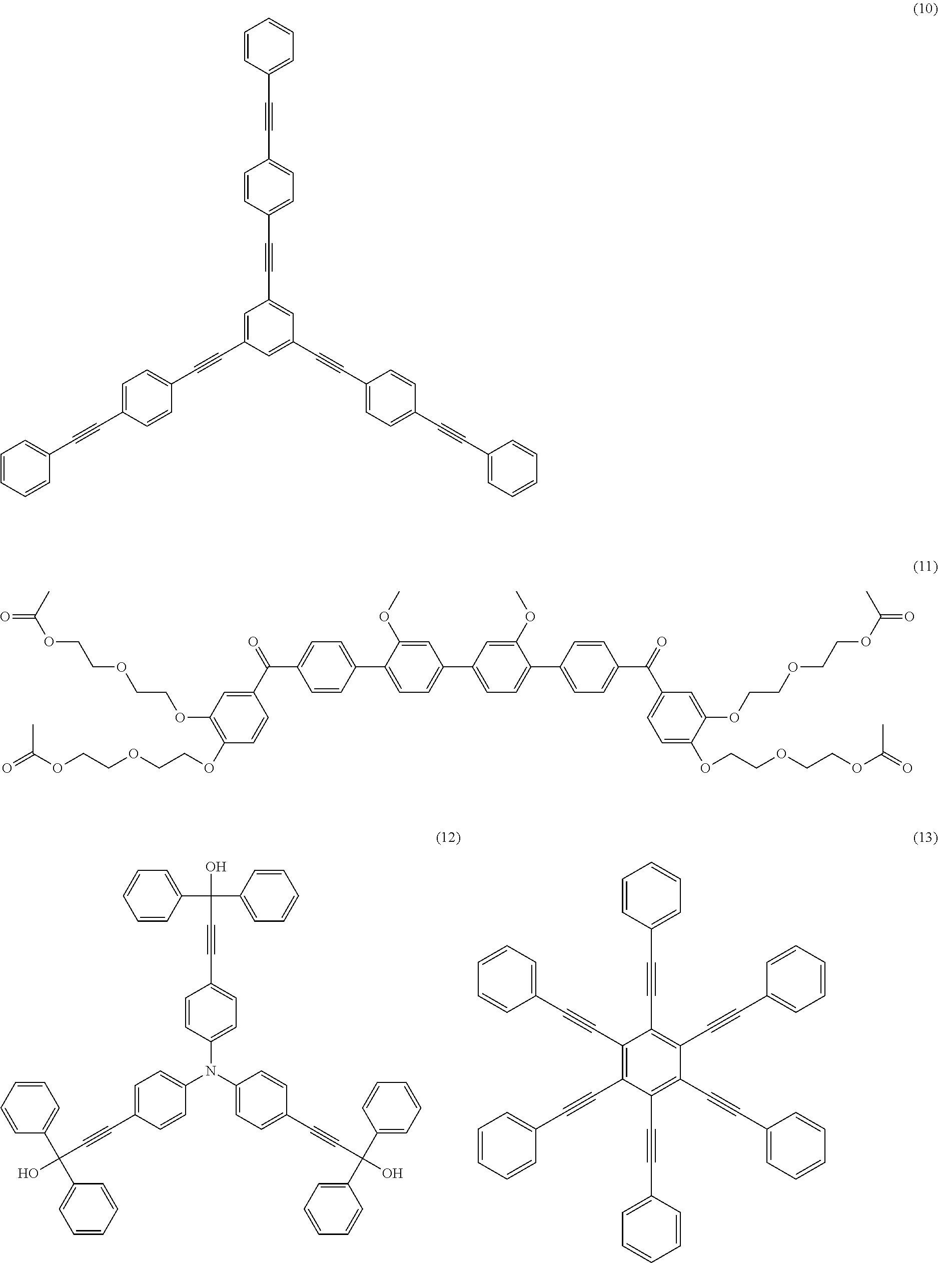

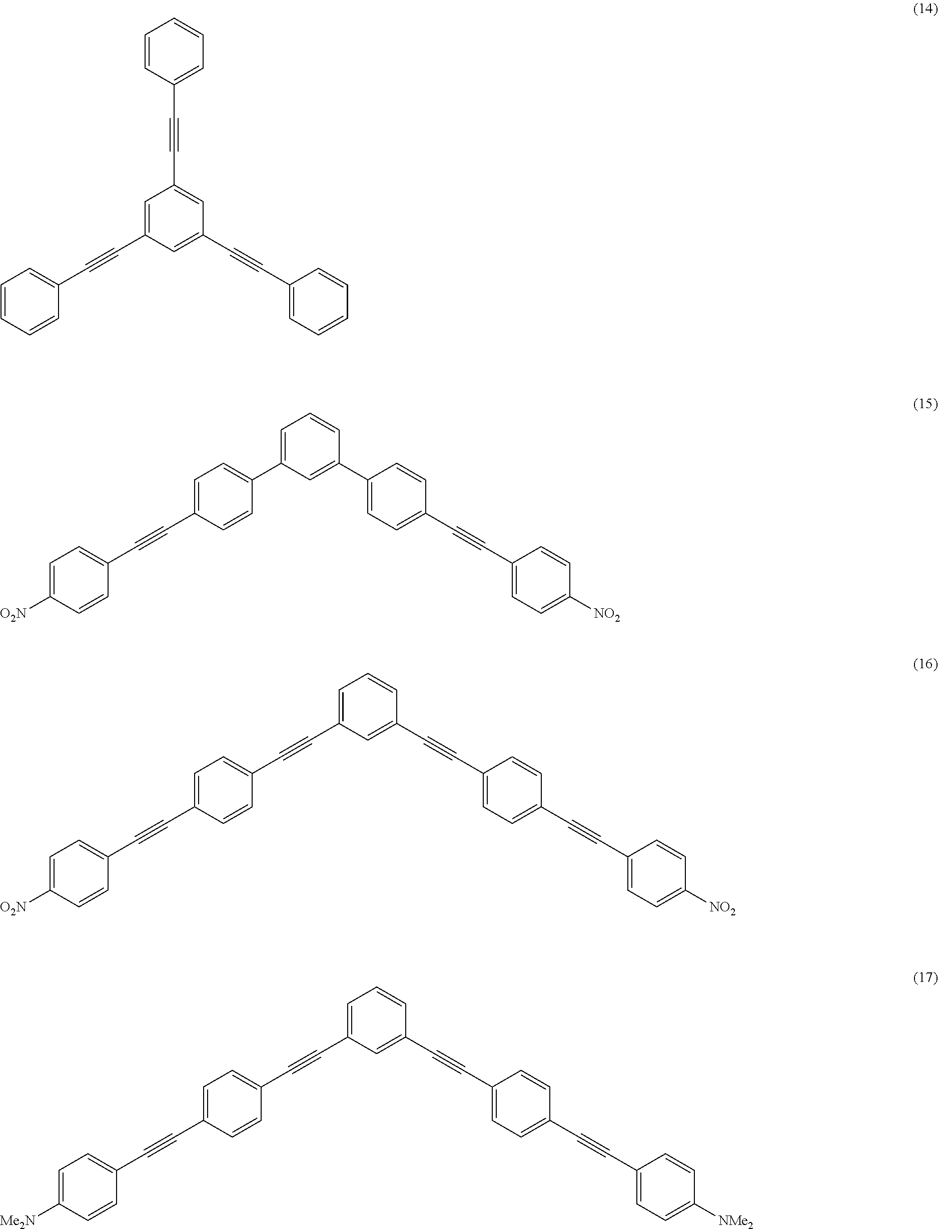

### Measurement of Two-Photon Absorption Cross-Sectional Area

Regarding Compound (2)-1, Compound (3)-1, and the compounds of Comparative examples 2 to 5, the two-photon absorption cross-sectional area with respect to the light having a wavelength of 405 nm was measured. The two-photon absorption cross-sectional area was measured using the Z-scan method described in J. Opt. Soc. Am. B, 2003, Vol. 20, p. 529. A titanium-sapphire pulse laser was used as the light source for measuring the two-photon absorption cross-sectional area. In particular, the second harmonic of the titanium-sapphire pulse laser was applied to the sample. The pulse width of the laser was 80 fs. The repetition frequency of the laser was 1 kHz. The average power of the laser was changed within the range of greater than or equal to 0.01 mW and less than or equal to 0.08 mW. The light from the laser was light having a wavelength of 405 nm. In particular, the light from the laser had a center wavelength of greater than or equal to 403 nm and less than or equal to 405 nm. The full width at half maximum of the light from the laser was 4 nm.

### Prediction of Two-Photon Absorption Cross-Sectional Area

Regarding the compounds presented in Table 1 to Table 6 above and compounds of Comparative examples 1, 3, and 6 to 8, the two-photon absorption cross-sectional area with respect to the light having a wavelength of 405 nm was predicted. In particular, the two-photon absorption cross-sectional area was calculated by the density functional theory method (DFT) calculation based on the second-order non-linear response theory described in J. Chem. Theory Comput. 2018, Vol. 14, p. 807. Regarding the DFT calculation, Turbomole version 7.3.1 (produced by COSMOlogic) was used as the software. Regarding the basis function, def2-TZVP was used. Regarding the functional, B3LYP was used.

When the two-photon absorption cross-sectional area was predicted, in advance, a calculation value and a measurement value of the two-photon absorption cross-sectional area of a known two-photon absorbing compound were specified, and linear regression was performed. A regression equation obtained by the linear regression was used, and the calculation value of the two-photon absorption cross-sectional area of the above-described compound was determined.

### Measurement of Molar Extinction Coefficient

Regarding Compound (2)-1, Compound (3)-1, and the compounds of Comparative examples 2 to 5, the molar extinction coefficient was measured by the method in conformity with the specification of JIS K 0115:2004. In particular, initially, a solution in which the compound was dissolved in a solvent was prepared as the measurement sample. The concentration of the compound in the solution was adjusted to 500 mmol/L. Subsequently, an absorption spectrum of the measurement sample was measured. The absorbance at a wavelength of 405 nm was read from the resulting spectrum. The molar extinction coefficient was calculated in accordance with the concentration of the compound in the measurement sample and the optical path length of the cell used for the measurement.

### Prediction of Molar Extinction Coefficient

Regarding the compounds presented in Table 1 to Table 6 above and compounds of Comparative examples 1, 3, and 6 to 8, the molar extinction coefficient was predicted. The DFT calculation was utilized for predicting the molar extinction coefficient. In particular, Gaussian 16 (produced by Gaussian) that is a quantum chemistry calculation program was used, and the excited state of the compound was calculated. In the excited state calculation, regarding the basis function, 6-31++G(d,p) was used. Regarding the functional, CAM-B3LYP was used. According to the excited state calculation, the energy for exciting the compound and the oscillator strength f were calculated. The oscillator strength has correlation to the molar extinction coefficient. Subsequently, the absorption spectrum is assumed to be Gaussian distribution, and the full width at half maximum was specified. In particular, the full width at half maximum was specified as 0.4 eV, and the absorption spectrum was drawn based on the absorption wavelength and the oscillator strength. The absorbance at a wavelength of 405 nm was read from the resulting spectrum. The absorbance was assumed to be a calculation value of the molar extinction coefficient.

When the molar extinction coefficient was predicted, in advance, a calculation value and a measurement value of the molar extinction coefficient of a known two-photon absorbing compound were specified, and linear regression was performed. A regression equation obtained by the linear regression was used, and the calculation value of the molar extinction coefficient of the above-described compound was determined.

### Measurement of Quantum Yield of Fluorescence

Regarding Compound (2)-1, Compound (3)-1, and the compounds of Comparative examples 2 to 4, the internal quantum yield of the fluorescence was measured. The measurement sample was prepared by dissolving the compound in the chloroform (CLF). An absolute PL quantum yield spectrometer (C9920-02 produced by Hamamatsu Photonics K.K.) was used for the measurement. The exciting wavelength was set to the peak wavelength of single-photon absorption of the compound. The measurement wavelength was appropriately adjusted within the range of greater than or equal to 350 nm and less than or equal to 650 nm not to overlap the absorption wavelength range of the compound. Regarding the reference, a CLF solvent was used.

The calculation value and the measurement value of the two-photon absorption cross-sectional area, the calculation value and the measurement value of the molar extinction coefficient ε (mol¹·L·cm⁻¹), the ratio σ/ε, and the quantum yield Φf(−) of the fluorescence are presented in Table 7 to Table 11. In Table 7 to Table 11, the ratio σ/ε was calculated in accordance with the measurement value of the two-photon absorption cross-sectional area and the measurement value of the molar extinction coefficient. Regarding the compound of which neither the measurement value of the two-photon absorption cross-sectional area nor the measurement value of the molar extinction coefficient was acquired, the ratio σ/ε was calculated in accordance with these calculation values. In Table 7 to Table 11, “No data” means that data were not acquired. As presented in Table 7 to Table 10, the compound of Example 1 is Compound (2)-1, the compound of Example 2 is Compound (3)-1, the compounds of Example 3 to Example 52 are Compound (2)-2 to Compound (2)-51, respectively, and the compounds of Example 53 to Example 88 are Compound (3)-2 to Compound (3)-51, respectively.

TABLE 7

| | | Two-photon absorption | | Molar extinction | | | |
|---|---|---|---|---|---|---|---|
| | | cross-sectional area | | coefficient | | | Quantum |
| | | σ (GM) | | ε (mol⁻¹ · L · cm⁻¹) | | | yield of |
| | | Measurement | Calculation | Measurement | Calculation | Ratio | fluorescence |
| | Compound | value | value | value | value | σ/ε | Φf (—) |
| Example 1 | (2)-1 | 380 | 600 | 0.02 | 0.7 | 19000 | 0.78 |
| Example 2 | (3)-1 | 1610 | 1320 | 13 | 18 | 124 | 0.79 |
| Example 3 | (2)-2 | No Data | 630 | No Data | 2.1 | 310 | No Data |
| Example 4 | (2)-3 | No Data | 550 | No Data | 1.4 | 400 | No Data |
| Example 5 | (2)-4 | No Data | 530 | No Data | 0.6 | 870 | No Data |
| Example 6 | (2)-5 | No Data | 620 | No Data | 2.0 | 300 | No Data |
| Example 7 | (2)-6 | No Data | 630 | No Data | 2.2 | 290 | No Data |
| Example 8 | (2)-7 | No Data | 650 | No Data | 1.6 | 420 | No Data |
| Example 9 | (2)-8 | No Data | 680 | No Data | 2.2 | 310 | No Data |
| Example 10 | (2)-9 | No Data | 490 | No Data | 0.5 | 900 | No Data |
| Example 11 | (2)-10 | No Data | 640 | No Data | 2.1 | 310 | No Data |
| Example 12 | (2)-11 | No Data | 520 | No Data | 1.3 | 410 | No Data |
| Example 13 | (2)-12 | No Data | 510 | No Data | 0.5 | 1060 | No Data |
| Example 14 | (2)-13 | No Data | 610 | No Data | 1.8 | 340 | No Data |
| Example 15 | (2)-14 | No Data | 640 | No Data | 2.3 | 280 | No Data |
| Example 16 | (2)-15 | No Data | 670 | No Data | 1.6 | 410 | No Data |
| Example 17 | (2)-16 | No Data | 710 | No Data | 2.4 | 300 | No Data |
| Example 18 | (2)-17 | No Data | 330 | No Data | 0.2 | 1560 | No Data |
| Example 19 | (2)-18 | No Data | 630 | No Data | 2.4 | 260 | No Data |
| Example 20 | (2)-19 | No Data | 620 | No Data | 2.1 | 300 | No Data |
| Example 21 | (2)-20 | No Data | 620 | No Data | 1.5 | 410 | No Data |
| Example 22 | (2)-21 | No Data | 640 | No Data | 2.0 | 310 | No Data |
| Example 23 | (2)-22 | No Data | 630 | No Data | 1.8 | 350 | No Data |
| Example 24 | (2)-23 | No Data | 630 | No Data | 1.6 | 400 | No Data |
| Example 25 | (2)-24 | No Data | 630 | No Data | 1.5 | 430 | No Data |
| Example 26 | (2)-25 | No Data | 620 | No Data | 1.9 | 320 | No Data |
| Example 27 | (2)-26 | No Data | 640 | No Data | 2.0 | 310 | No Data |
| Example 28 | (2)-27 | No Data | 510 | No Data | 0.9 | 560 | No Data |
| Example 29 | (2)-28 | No Data | 450 | No Data | 0.4 | 1070 | No Data |
| Example 30 | (2)-29 | No Data | 600 | No Data | 2.2 | 280 | No Data |

TABLE 8

| | | Two-photon absorption | | Molar extinction | | | |
|---|---|---|---|---|---|---|---|
| | | cross-sectional area | | coefficient | | | Quantum |
| | | σ (GM) | | ε (mol⁻¹ · L · cm⁻¹) | | | yield of |
| | | Measurement | Calculation | Measurement | Calculation | Ratio | fluorescence |
| | Compound | value | value | value | value | σ/ε | Φf (—) |
| Example 31 | (2)-30 | No Data | 630 | No Data | 2.4 | 260 | No Data |
| Example 32 | (2)-31 | No Data | 690 | No Data | 1.9 | 360 | No Data |
| Example 33 | (2)-32 | No Data | 770 | No Data | 3.3 | 240 | No Data |
| Example 34 | (2)-33 | No Data | 430 | No Data | 0.6 | 740 | No Data |
| Example 35 | (2)-34 | No Data | 740 | No Data | 3.4 | 210 | No Data |
| Example 36 | (2)-35 | No Data | 800 | No Data | 3.9 | 210 | No Data |
| Example 37 | (2)-36 | No Data | 630 | No Data | 1.9 | 320 | No Data |
| Example 38 | (2)-37 | No Data | 920 | No Data | 5.9 | 160 | No Data |

TABLE 9

| | | Two-photon absorption | | Molar extinction | | | |
|---|---|---|---|---|---|---|---|
| | | cross-sectional area | | coefficient | | | Quantum |
| | | σ (GM) | | ε (mol⁻¹ · L · cm⁻¹) | | | yield of |
| | | Measurement | Calculation | Measurement | Calculation | Ratio | fluorescence |
| | Compound | value | value | value | value | σ/ε | Φf (—) |
| Example 39 | (3)-2 | No Data | 1350 | No Data | 20.7 | 70 | No Data |
| Example 40 | (3)-3 | No Data | 1360 | No Data | 23.2 | 60 | No Data |
| Example 41 | (3)-4 | No Data | 1320 | No Data | 23.6 | 60 | No Data |
| Example 42 | (3)-5 | No Data | 1300 | No Data | 19.2 | 70 | No Data |
| Example 43 | (3)-6 | No Data | 1340 | No Data | 23.7 | 60 | No Data |
| Example 44 | (3)-7 | No Data | 1370 | No Data | 20.9 | 70 | No Data |
| Example 45 | (3)-8 | No Data | 1410 | No Data | 24.1 | 60 | No Data |
| Example 46 | (3)-9 | No Data | 1400 | No Data | 25.5 | 50 | No Data |
| Example 47 | (3)-10 | No Data | 1370 | No Data | 20.7 | 70 | No Data |
| Example 48 | (3)-11 | No Data | 1380 | No Data | 23.3 | 60 | No Data |
| Example 49 | (3)-12 | No Data | 1310 | No Data | 24.4 | 50 | No Data |
| Example 50 | (3)-13 | No Data | 1270 | No Data | 18.0 | 70 | No Data |
| Example 51 | (3)-14 | No Data | 1360 | No Data | 24.7 | 60 | No Data |
| Example 52 | (3)-15 | No Data | 1410 | No Data | 21.5 | 70 | No Data |
| Example 53 | (3)-16 | No Data | 1460 | No Data | 25.3 | 60 | No Data |
| Example 54 | (3)-17 | No Data | 1450 | No Data | 27.6 | 50 | No Data |
| Example 55 | (3)-18 | No Data | 1330 | No Data | 22.8 | 60 | No Data |
| Example 56 | (3)-19 | No Data | 1340 | No Data | 22.4 | 60 | No Data |
| Example 57 | (3)-20 | No Data | 1350 | No Data | 23.2 | 60 | No Data |
| Example 58 | (3)-21 | No Data | 1340 | No Data | 23.1 | 60 | No Data |
| Example 59 | (3)-22 | No Data | 1330 | No Data | 21.5 | 60 | No Data |
| Example 60 | (3)-23 | No Data | 1330 | No Data | 20.4 | 70 | No Data |
| Example 61 | (3)-24 | No Data | 1330 | No Data | 20.2 | 70 | No Data |
| Example 62 | (3)-25 | No Data | 1320 | No Data | 26.1 | 50 | No Data |
| Example 63 | (3)-26 | No Data | 1370 | No Data | 21.0 | 70 | No Data |
| Example 64 | (3)-27 | No Data | 1390 | No Data | 24.7 | 60 | No Data |
| Example 65 | (3)-28 | No Data | 1300 | No Data | 24.7 | 50 | No Data |
| Example 66 | (3)-29 | No Data | 1280 | No Data | 21.3 | 60 | No Data |

TABLE 10

| | | Two-photon absorption | | Molar extinction | | | |
|---|---|---|---|---|---|---|---|
| | | cross-sectional area | | coefficient | | | Quantum |
| | | σ (GM) | | ε (mol⁻¹ · L · cm⁻¹) | | | yield of |
| | | Measurement | Calculation | Measurement | Calculation | Ratio | fluorescence |
| | Compound | value | value | value | value | σ/ε | Φf (—) |
| Example 67 | (3)-30 | No Data | 1360 | No Data | 24.8 | 50 | No Data |
| Example 68 | (3)-31 | No Data | 1440 | No Data | 22.5 | 60 | No Data |
| Example 69 | (3)-32 | No Data | 1550 | No Data | 28.3 | 50 | No Data |
| Example 70 | (3)-33 | No Data | 1450 | No Data | 30.2 | 50 | No Data |
| Example 71 | (3)-34 | No Data | 1500 | No Data | 28.1 | 50 | No Data |
| Example 72 | (3)-35 | No Data | 1600 | No Data | 30.5 | 50 | No Data |
| Example 73 | (3)-36 | No Data | 1330 | No Data | 20.1 | 70 | No Data |
| Example 74 | (3)-37 | No Data | 1770 | No Data | 30.5 | 60 | No Data |

TABLE 11

| | | Two-photon absorption | | Molar extinction | | | |
|---|---|---|---|---|---|---|---|
| | | cross-sectional area | | coefficient | | | Quantum |
| | | σ (GM) | | ε (mol⁻¹ · L · cm⁻¹) | | | yield of |
| | | Measurement | Calculation | Measurement | Calculation | Ratio | fluorescence |
| | Compound | value | value | value | value | σ/ε | Φf (—) |
| Comparative | (10) | No Data | 2350 | No Data | 170 | 15 | No Data |
| example 1 | | | | | | | |
| Comparative | D-29 (11) | 570 | No Data | 35 | No Data | 15 | 0 |
| example 2 | | | | | | | |
| Comparative | 1f (12) | 380 | 370 | 70 | 60 | 5 | 0.3 |
| example 3 | | | | | | | |
| Comparative | hexakis(phenyl- | 23000 | No Data | 2370 | No Data | 10 | greater than |
| example 4 | ethynyl)benzene (13) | | | | | | or equal to |
| | | | | | | | 0.1 and less |
| | | | | | | | than or |
| | | | | | | | equal to 0.3 |
| Comparative | 1,3,5-tri(phenyl- | 110 | No Data | 10 | No Data | 10 | No Data |
| example 5 | ethynyl)benzene (14) | | | | | | |
| Comparative | (15) | No Data | 640 | No Data | 48.2 | 10 | No Data |
| example 6 | | | | | | | |
| Comparative | (16) | No Data | 1040 | No Data | 103.9 | 10 | No Data |
| example 7 | | | | | | | |
| Comparative | (17) | No Data | 1590 | No Data | 158.0 | 10 | No Data |
| example 8 | | | | | | | |

As clearly presented in Table 7 to Table 11, all of the compounds of the examples corresponding to Compound A that are denoted by Formula (1) and that satisfy requirements (a) and (b) had a larger value of the ratio σ/ε with respect to the light having a wavelength of 405 nm than the compounds of the comparative examples. It was found from this result that Compound A above had high nonlinearity in light absorption with respect to the light having a wavelength in a short wavelength range and that the non-linear optical absorption characteristics were improved. Further, the compounds of Examples 1 and 2 also had fluorescent characteristics. On the other hand, the compounds of Comparative examples 1 to 8 tended to have a smaller two-photon absorption cross-sectional area a or have a larger molar extinction coefficient ε than the compounds of the examples. Consequently, the ratio σ/ε of the compounds of Comparative examples 1 to 8 had a small value.

As is clear from Formula (1), Compound A has a V-shaped molecular skeleton. It is understood from comparison between Example 2 and Comparative examples 1 and 5 that a V-shaped molecular skeleton is suitable for improving the ratio σ/ε compared with a three-branched molecular skeleton. That is, it is estimated that the ratio σ/ε of Compound A with respect to the light having a wavelength of 405 nm had a small value due to the V-shaped molecular skeleton.

Further, as Reference example 1, regarding the compound in which R¹ in Formula (2) represents a nitro group and each of R² to R²² represents hydrogen atom, the two-photon absorption cross-sectional area and the molar extinction coefficient were calculated, and the value of the ratio σ/ε was calculated. In Reference example 1, the value of the resulting ratio σ/ε was 130. In addition, as Reference example 2, regarding the compound in which R¹ in Formula (2) represents a dimethylamino group and each of R² to R²² represents hydrogen atom, the two-photon absorption cross-sectional area and the molar extinction coefficient were calculated, and the value of the ratio σ/ε was calculated. In Reference example 2, the value of the resulting ratio σ/ε was 120. It is clear from, for example, comparisons between Example 3, Example 11, Example 19, and Example 27 and Reference examples 1 and 2 that when at least one of R² to R²² represents a substituent other than a hydrogen atom in Compound A and when the Hammett substituent constant 6p of the substituent being within the range of greater than or equal to −0.2 and less than or equal to 0.2, the value of the ratio σ/ε tended to be large. The cause of this is conjectured that the substituent having a substituent constant 6p within the range of greater than or equal to −0.2 and less than or equal to 0.2 has a low electron-withdrawing property or a low electron donating-property. In particular, the substituent having a low electron-withdrawing property or a low electron-donating property enables the energy gap between HOMO and LUMO to be suppressed from decreasing. Consequently, the peak due to single-photon absorption can be suppressed from shifting to a long wavelength, and the ratio σ/ε with respect to the light having a wavelength in a short wavelength range can be suppressed from decreasing. Actually, in Examples 3 to 34, the value of the molar extinction coefficient determined by the DFT calculation tended to be small compared with that in Examples 35 to 48 and was substantially equal to Example 1 with no substitution.

In this regard, in Examples 3 to 10 and the like, the substituent constant σₚ of a methyl group was −0.17. In Examples 11 to 18 and the like, the substituent constant σₚ of a propyl group was −0.13. In Examples 19 to 26 and the like, the substituent constant σₚ of a fluoro group was 0.06. In Examples 27 to 34 and the like, the substituent constant σₚ of a trimethylsilyl group was −0.07. In Reference example 1, the substituent constant σₚ of a nitro group was 0.78. In Reference example 2, the substituent constant σₚ of an amino group was −0.83.

The compound according to the present disclosure can be utilized for uses such as a recording layer of a three-dimensional optical memory, a photo-curable resin composition for three-dimensional (3D) laser microfabrication, and the like. The compound according to the present disclosure has light-absorption characteristics that exhibit high nonlinearity with respect to the light having a wavelength in a short wavelength range. Consequently, the compound according to the present disclosure can realize very high spatial resolution in uses such as a three-dimensional optical memory, a shaping machine, and the like. Further, The compound according to the present disclosure tends to have a high quantum yield of fluorescence. Consequently, the compound being utilized for a recording layer of a three-dimensional optical memory enables a system in which an ON/OFF state of the recording layer is read in accordance with a change in the fluorescence from the compound to be adopted. The compound according to the present disclosure can also be used as a fluorochrome material used for a two-photon fluorescent microscope and the like. The compound according to the present disclosure can preferentially realize two-photon absorption rather than single-photon absorption even when laser light having low light intensity is applied, compared with a compound in the related art.

What is claimed is:

1. A compound represented by Formula (1) below, 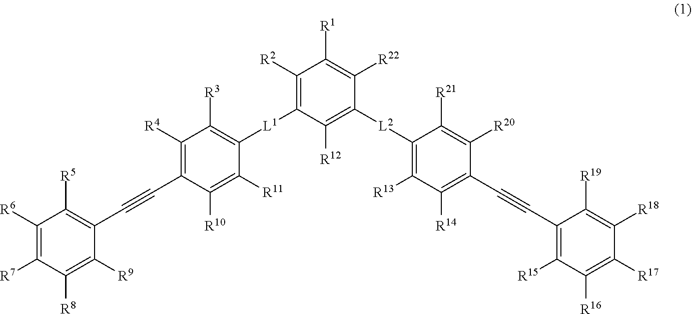
in Formula (1) above,
R¹ to R²² each independently contain at least one atom selected from the group consisting of H, B, C, N, O, F, Si, P, S, Cl, I, and Br,
L¹ and L² each independently represent a single bond or —C≡C—,
R¹, R², R⁶, R⁷, R¹², R¹⁷, and R²² are each independently a substituent other than a substituent having an aromatic ring, and
R¹ to R²² are each independently a hydrogen atom or a substituent having a Hammett substituent constant σₚ within a range of greater than or equal to −0.2 and less than or equal to 0.2.

2. The compound according to claim 1, wherein the compound is represented by Formula (2) below,

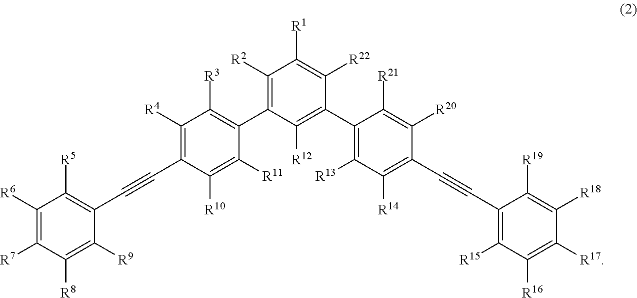

3. The compound according to claim 1, wherein the compound is represented by Formula (3) below,

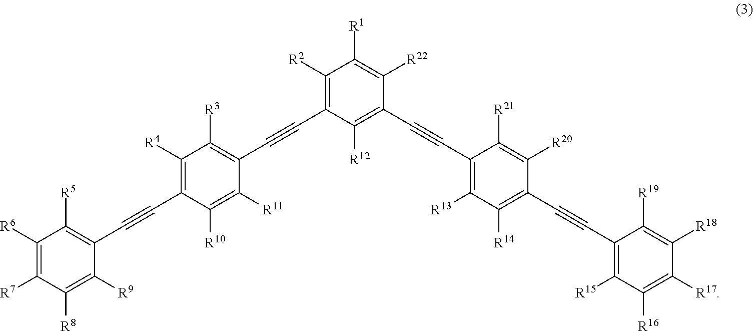

4. The compound according to claim 1,
wherein R¹ to R²² are each independently a hydrogen atom, a halogen atom, a hydrocarbon group, a halogenated hydrocarbon group, a substituent containing an oxygen atom, a substituent containing a nitrogen atom, a substituent containing a sulfur atom, a substituent containing a silicon atom, a substituent containing a phosphorus atom, or a substituent containing a boron atom.

5. The compound according to claim 1,
wherein R¹ to R²² are each independently a hydrogen atom.

6. The compound according to claim 1,
wherein the compound is used for a device that utilizes light having a wavelength of greater than or equal to 390 nm and less than or equal to 420 nm.

7. An optical absorbing material comprising:
the compound according to claim 1.

8. A non-linear optical absorbing material comprising:
the compound according to claim 1.

9. A recording medium comprising:
a recording layer containing the compound according to claim 1.

10. An information recording method comprising;
preparing a light source that emits light having a wavelength of greater than or equal to 390 nm and less than or equal to 420 nm, and
condensing the light from the light source and applying the light to the recording layer of the recording medium according to claim 9.

11. An information reading method, the information being recorded by the information recording method according to claim 10, comprising:
measuring an optical characteristic of the recording layer by applying light to the recording layer of the recording medium, and
reading the information from the recording layer.

12. The information reading method according to claim 11,
wherein the optical characteristic is intensity of fluorescence emitted from the recording layer.
